(19) United States
(12) Patent Application Publication
(10) Pub. No.: US 2018/0056022 A1
(43) Pub. Date: Mar. 1, 2018

(54) HAND-ACTUATED DRY POWDER INHALER AND ITS USE

(71) Applicants: James Zhou LIU, Milford, DE (US); Yuehua GU, Kendall Park, NJ (US)

(72) Inventors: James Zhou LIU, Milford, DE (US); Yuehua GU, Kendall Park, NJ (US)

(21) Appl. No.: 15/560,319

(22) PCT Filed: Mar. 18, 2016
(86) PCT No.: PCT/US16/23058

§ 371 Date: Sep. 21, 2017

(51) Int. Cl. A61M 16/14; A61M 15/00; A61K 9/00; A61K 9/14

(52) U.S. Cl. CPC A61M 16/14; A61M 15/0021; A61M 15/0086; A61M 15/0035; A61M 2202/064; A61K 9/0075; A61K 9/14; A61M 2205/075; A61M 15/0045

(57) ABSTRACT

Disclosed herein are inhalers, having an airflow chamber (8) for receiving air from an inlet channel and holding a medicament in powder form, the chamber (8) forming a starting section of an air passage (4) and including a turbulence generating mechanism that generates turbulent air flow through the chamber (8) such that during use of the inhaler, the air flow causes vibration of a holder (31) received by the chamber (8) to assist in releasing the medicament; a positive pressure supplier (2) connected to the chamber (8) for triggering and driving the medicament through the air passage towards a mouthpiece (6): a closure means (3.1, 3.2) that closes the airflow chamber (8) to form, an air passage; a piercing element (1) coupled to the airflow chamber (8) to pierce the holder and release the medicament into the air passage; and an outlet air channel that connects to the chamber (8).

### CROSS-REFERENCE TO RELATED APPLICATIONS

The present application claims priority to and the benefit of U.S. Provisional Application No. 62/136,483, filed Mar. 21, 2015. The aforementioned priority application is incorporated herein by reference, in its entirety.

### TECHNICAL FIELD

The present application relates to inhaler devices, systems, and methods for delivery of powders via inhaler devices.

### BACKGROUND

Disclosed herein are inhaler devices and methods that seek to painlessly deliver fine powder, accurately and directly to the human respiratory tract, through a hand, actuated positive pressure action combined with inhalation by the user. Presently, known dry powder inhalers (DPIs) are breath-actuated only. The function of these DPIs is dependent on its flow rate under breath-actuation by different end users. More specifically, the delivered dose of the medicament is dependent on the flow rate of the DPIs while operated by the individual user. There is a long felt need for a DPI device that, while operated by different users under, normal conditions, delivers substantially the same amount of medicament to the respiratory tract from the same dry powder inhaler. Embodiments disclosed herein seek to resolve these issues related to DPI delivery. A safe and effective medical device for the administration of medicine for the treatment of human diseases or disorders plays a key role in advancing the effectiveness of both traditional and modem medicines. Traditional Chinese medicines (TCM) have been used for thousands of years in China and other countries, but there is no TCM natural medicine administered by using a DPI. Only a limited, number of modern medicines have been delivered through non-invasive inhalation due to the limitation of the available DPIs. Embodiments disclosed herein are directed to medical devices for the administration of a drug to treat a human disease. These medical devices allow for direct delivery of numerous new compositions into the human lower respiratory tract. One such medical devices is a hand-actuated positive pressure dry powder inhaler (PP-DPI). Embodiments of the invention disclosed, herein also includes kits for treating asthma, diabetes, obesity, erectile dysfunction and other diseases or disorders by delivering the right amount of TCMs or modem medicine using a new PP-DPI into the human respiratory tract. A fine powder delivered painlessly, easily, accurately and directly to the respiratory tract through an effortless non-breath-actuated inhaler has many advantages as compared to oral intake or injection. Embodiments of the invention disclosed herein thus greatly enhances existing DPI drug delivery and advances traditional and modem medicines.

An effective and safe therapy relies on an appropriate drug and its delivery. For treating respiratory diseases, inhalation is a non-invasive approach for drug administration. For treating many non-respiratory disease or disorder systemically, inhalation of the drug to the lungs is quick, easy and painless. Many dry powder inhalers (DPIs) have been developed because of their advantages over known metered dose inhalers (MDIs). DPIs in general are safer than MDIs and cause fewer irritations. The active ingredient(s) can be directly inhaled into the lungs with a DPI without using any preservatives or propellant.

Conventional DPIs, however, have many disadvantages, and hence limited their usefulness to become the most common device for drug administration. Although there are numerous differences among existing DPIs and enhancements, prior art DPIs belong to the “breath-actuated DPI” category. The dosages and delivery efficacy of these breath-actuated DPIs are impacted by their breath based actuation mechanism, There is a critical need to reduce the flow rate dependence of breath-actuated DPIs, and, specifically, the flow rate dependence of the delivered dose of the medicament they deliver. In particular, there is a need to ensure that different patients receive substantially the same delivered dose from the same breath-actuated DPI. Embodiments disclosed herein overcome the limitations of existing DPIs, dramatically change the nature of the DPI delivery when operated by individual patients, and, among numerous other advantages, enhance direct drug delivery into the human lower respiratory tract and advance traditional and modern medicines.

Traditional Chinese medicine (TCM) has a history of several thousand years, Its origin could be traced back to remote antiquity. An important aspect of the TCM, herbal medicine, has been developed to cure many diseases and evolved into a unique and integrated theoretical system. Historically, there are roughly 13,000 medicines used in TCM and over 100,000 medicinal recipes recorded in the literature. Currently, TCM is an important part of medicine practiced in China and many other countries. The most common route of administration of the TCM therapies is through oral intake. Injection, patch, spray and other forms of administrations of TCM therapies have also been developed. To further improve the effectiveness of TCM, a more efficient delivery device for administration of the TCM therapy is disclosed herein.

It is known that, for the same modem medicine, e.g., epinephrine, oral intake is less efficient compared to direct administration, even in a much smaller amount, to the lower respiratory tract to treat allergy or respiratory diseases The same is true for the TCM therapeutic formulae. Since the delivery of certain modern drugs to the lungs is in practice using the existing breath-actuated DPIs or MDIs, the delivery of certain, appropriately prepared TCM formulae can also be carried out efficiently by using the embodiments of PP-DPI disclosed herein.

It is beneficial to review the existing inhalation devices herein. A metered dose inhaler (MDI) is a handheld device that delivers a specific amount of medication in an aerosol form to the lower respiratory tract. The MDI includes a pressurized canister inside a plastic case, with a mouthpiece attached. With an MDI, the user presses on the device while inhaling the medication directly into the lungs. Its portability makes it easy to use anywhere, anytime. A dry powder inhaler (DPI) is also a handheld device that delivers a specific amount of medication in an aerosol form to the lower respirator) tract. The DPI includes a chamber/housing to hold the active ingredient in a fine powder form, with an air passage connected to, the mouthpiece, DPIs can have, a chamber for loading a single dose, or have an internal reservoir containing multiple doses that are metered by the device itself during actuation by the patient. Its portability also makes it easy to use anywhere, anytime. Since known current DPIs are driven by the negative pressure generated by the patient's inhalation, so called breath-actuated inhalation, these DPIs are classified as the negative pressure dry powder inhalers (NP-DPIs). This is to differentiate from the positive pressure dry powder inhaler (PP-DPI) in embodiments of invention, as disclosed herein.

Existing NP-DPIs have limitations. Specifically, the inconsistency of the inspiratory flow rates of NP-DPI drug delivery when operated by different patients is a significant disadvantage. The inspiratory rate required to deliver the medication in MDI inhalers is about 30 L/min, while the rate required for the NP-DPIs is much higher (ranging from 30-120 L/min) and differs based on the build of the inhaler, as well as the different inhaling capacity of the patients. This higher inspiratory rate would make it more difficult for small children or even adults, who have a lower inspiration capacity of the lungs, to receive the medication properly. Using NP-DPIs also requires the patient to be careful not to disperse the medication via exhalation into the device prior to using.

Wachter et al. (U.S. Pat. No. 6,085,742) (hereinafter “Wachter”) teaches an inhalation device for administering a medicament within a prescribed dosage range to avoid over or under administration. However, Wachter refers to a breath-actuated DPI. In Wachter air-pressure is applied as a fraction of the patient's inspiration volume. It is very difficult for the patient to introduce the gas-bolus after the patient actuated the DPI with breath. Since its filing in 1997, a device according to the Wachter invention has not been known to be made commercially viable, probably due to its limitations. In contrast, embodiments disclosed herein provide a hand-triggered positive pressure actuated DPI, which can generate the pre-measured dose of therapeutic aerosols for ready inhalation by the patient.

Existing NP-DPIs have additional disadvantages: because of the wide range of DPI designs there are challenges in developing uniform information and common instructions, regarding the appropriate use of the DPI as a whole category. Variations among aerosol formulation also create challenges to the patient use and caregiver instructions. NP-DPIs are also more susceptible to contamination because of their bi-directional flow design.

For improving the performance of any NP-DPI, the use of a carrier with the right particle size is generally needed. Since the active ingredient in a very fine powder form in general has poor flow properties, it is a good practice to incorporate a coarse carrier within the formulation to increase the overall flowability of the powder. It has been reported that larger carrier particles normally exhibit larger surface discontinuities than fine crystals. This may have the advantage of providing shelter to drug particles from the press-on forces during the mixing and homogenization process, as the drug particles tend to assemble in these discontinuities during mixing. Therefore, a high carrier particle size does not necessarily have a negative effect on the drug deposition profiles after inhalation. In one study. formulations of inspirable recombinant human granulocyte- colony stimulating factor with larger carriers (90-125 micron) showed a higher drug dispersion than the same formulation with 38-75 micron carriers, and it is interpreted that this is due to the lower inter-particle forces among the larger sized particles (French et al. 1996). Similar results were shown for enhanced inhalation performance of terbutaline sulphate from a formulation containing coarse lactose (53-105 micron) than the same drug containing fine lactose carriers with size less than 53 micron (Byron et al., 1990). Embodiments of the PP-DPI, as disclosed herein, overcome the above described disadvantages of the NP-DPIs.

It is also beneficial to briefly review and compare current methods for non-inhalation versus inhalation based drug delivery. Oral delivery of any absorbable drug has its obvious advantages, and even for big molecules, such as insulin, scientists tried to make special microcapsules to by-pass the gastrointestinal tract's destruction. However if the therapeutic effect has time sensitivity, the slower drug effect of oral intake due to the long time needed to pass through the gastrointestinal tract before being metabolized in the liver is not desirable. A quick relief of the symptom is often preferred by a patient. Patients with normally functional lungs will find it easy to breathe in a powder formulation for a quick therapeutic effect, even using a NP-DPI. With the accuracy of drug dose delivery secured by using a new PP-DPI, such as shown and described with respect to embodiments of the invention disclosed herein, more patients will benefit from using such a device to receive the medicine they need directly into the lungs. The inhaled medicine in general works faster than the same medicine in a pill. In addition, the patients will take less medicine than what would be necessary via an oral delivery. Hence, many side effects can be reduced or even eliminated due to the smaller exposure of the given drug material. Therefore, PP-DPI delivery will offer many more advantages than swallowing the pill.

It is well known that drug injection has many advantages and disadvantages. The accurate dose and the quick effect caused by the injected drug are obvious. The disadvantages are related to overcoming the difficulties and expenses in manufacturing injectable drugs, maintaining their shelf-life during storage and transportation for the pharmaceutical companies and retail operations. And for the patient, the direct pain during the injection, the risk of infection, the high cost of the drug and the cost of receiving the injection from a medical professional, are obvious disadvantages. There is sufficient evidence to support the preference of the inhalation, route over injection routes, when both are viable. For treating many respiratory diseases, inhaled drugs are locally targeting the therapeutic organ, which generally allows for a lower dose than is necessary with systemic delivery (oral or injection), and thus fewer and less severe adverse effects are anticipated. Hence, new PP-DPIs as described with respect to embodiments of the invention is expected to meet a long felt need in the pharmaceutical and health maintenance industry for a medical device which provides a safer and more effective delivery of therapy.

It is also helpful to review several diseases and their current therapies. Many respiratory diseases have a complicated etiology and pathogenesis. Asthma is an example. As defined by the Global Initiative for Asthma in 2008 as part of the Global Strategy for Asthma Management and Prevention (Bateman 2008), “asthma is a chronic inflammatory disorder of the airways in which many cells and cellular elements play a role during the pathogenesis. The chronic inflammation is associated with airway hyper-responsiveness that leads to recurrent episodes of wheezing, breathlessness, chest tightness, and coughing, particularly at night or in the early morning. These events are usually associated with widespread, but variable, airflow obstruction within the lung that is often reversible either spontaneously or with treatment.” From this definition, it is clear that multiple therapeutic agents should be utilized in order to effectively control asthma.

Different types of asthma require different therapies. Infection triggered asthma and allergen triggered asthma are the most common types, but neither kind of asthma can be preemptively prevented or treated in a conventional manner. Antiviral drugs that treat colds do not presently exist, hence virus triggered asthma cannot be chemically prevented. For allergen triggered asthma, unless the specific allergen is known and can be avoided, or a vaccine is developed, or a specific immunotherapy is used, allergy-related asthma largely cannot be prevented, either.

Similar to treating a cancer using a number of chemotherapies, combinational therapies are needed for treating asthma and other diseases/disorders. Many current, asthma therapies target a single pathogenic process, such as using epinephrine to dilate bronchi, or corticosteroid (steroid) to suppress inflammation. While inhaled steroids help prevent/reduce asthma symptoms, they do not relieve asthma symptoms during an attack. The opposite is also true: epinephrine can only dilate bronchi during an asthma attack but is unable to reduce inflammation between asthma attacks. To overcome both drugs' limitation, Advair® was developed as a combinational drug that includes an anti-inflammation agent, fluticasone propionate, and a long-acting bronchodilator, salmeterol xinafoate. Salmeterol is a long-acting beta2-adrenergic receptor agonist drug which causes bronchodilation by relaxing the smooth muscle in the airway so as to treat the exacerbation of asthma. This drug is used when long-term medication, such as inhaled corticosteroids, does not control breathing problems. When the two chemical drugs are used together. their side effects become more complicated. But evaluating the benefit/risk ratio, combinational drugs are better than a single drug. There are several other examples.

To better manage asthma, without introducing additional side effects, simultaneous application of multiple functional ingredients is needed. Embodiments of the invention disclosed herein overcome this challenge by including the current modem therapy plus a number of herbs which have been successfully used in the TCM for hundreds of years for managing asthma. and the herb preparations can be delivered by using a dry powder inhaler.

Asthma was recognized in ancient China and there are a number of established, classical formulae used in TCM practice. Ma Huang is an herb which has been used in TCM for treating asthma. Now, several modern asthma drugs are made with ephedrine, a substance extracted from Ma Huang, an herb. Because these drugs are orally ingested, the effect take place quickly. However, if it is inhaled directly to the lower respiratory tract, its effect would be much fast. In recent years, researchers have begun to investigate the potential use of some of these herbal formulae and suggested that there is potential for the development of herbal interventions for asthma. Since 2005, there have been a number of publications reporting double-blind, placebo-controlled clinical studies investigating these Chinese herbal formulae in controlling asthma symptoms. A few of these studies are summarized below.

In 2007, Hoang et al. reported the impact of a Sophora flavescens Ait (Ku Shen) extract with excitatory modulator activity in the management of asthma (Phytother Res. 2007 June; 21(6):554-7). An open and selective 3-year follow-up study with 14 chronic refractory asthmatics aged 22 to 70 was conducted. Participants received an extract of Sophora flavescens water-extract powder in capsules with a dose equal to 4 g of dried root three times daily for 3 months, and 2 times daily for 6 months and once daily for 27 months thereafter. Medication use, a diary of symptoms and respiratory function were recorded. The test subjects' quality of life, clinical symptoms and respiratory function measurements were recorded. This study showed that the use of the inhaled corticosteroid and beta-agonists were reduced or eliminated during the study period. No significant adverse reactions were reported. The authors have concluded that the extract of Sophora flavescens appears to be a safe and effective alternative treatment for refractory chronic asthma. In an animal model of asthma, Sophora flavescens alone reduced allergic airway responses, as reported by Wen et al. in J Allergy Clin Immunol (Abstract) 2004; 113:218.

In 2005, Hsu et al. reported in the journal of Pediatr Allergy Immunol 2005; 16:76-81 the encouraging results of a clinical investigation of a complementary TCM therapy for asthma. Their study evaluated the efficacy and safety of a Chinese herbal formula modified Mai Men Dong Tang for the treatment of persistent, mild-to-moderate asthma. Modified Mai Men Dong Tang (mMMDT) includes 5 Chinese herbs, Radix Glycyrrhizae, Radix Ophiopogonis), Radix Panacis Quinquefolii). Tuber Pinellia), and Herba Tridacis Procumbentis. This four-month trial included 100 asthmatics aged 5 to 18. The two active groups in the trial received 40 mg modified Mai Men Dong Tang (40 patients), or 80 mg modified Mai Men Dong Tang (40 patients) respectively. The control group received placebo capsules (20 patients). Western medications as a part of a standard asthma therapy were provided equally to all groups, although it is unclear if there were adjustments for severity of disease at the baseline. Parameters used to evaluate efficacy were changes in FEV1, symptom score. total serum IgE and dust-mite-specific IgE. Safety assessments included complete blood count, and liver and kidney function tests. Relative to the baseline, significantly greater increases in FEV1 were demonstrated for both modified Mai Men Dong Tang-treated groups in comparison with the placebo group (P<0.05 for both doses of modified Mai Men Dong Tang). Symptom scores were similarly improved in both modified Mai Men Dong Tang treatment groups. The serum total IgE for the 80 mg/day dose of modified Mai Men Dong Tang treatment showed a decreasing tendency but no statistically significant difference was found. No drug-related adverse effects were reported. Possible efficacy of modified Mai Men Dong Tang as a monotherapy for asthma has not been tested.

In 2006 Chan et al. reported in Pediatr Allergy Immunol 2006; 17:316-22 that Ding Chuan Tang (DCT), a Chinese herbal decoction, could reduce airway hyper responsiveness (AHR) in stabilized asthmatic children in a randomized, double-blind clinical trial. The DCT decoction was made with 9 herbs. This study enrolled children 8-15 years of age, who were diagnosed to have mild-to-moderate persistent asthma. They were randomly assigned to receive 6.0 g DCT or placebo daily for 12 weeks. The self-recorded daily symptom scores, medication scores, and morning and evening peak expiratory flow rates were returned during monthly office visits. Pulmonary function testing, methacholine challenge testing, and serum inflammatory mediators were measured at baseline and at the end of the trial. Fifty-two asthmatic children completed the clinical study. Twenty-eight patients in the treatment group and 24 in the placebo, group completed the study. At the end of the treatment period, AHR determined by log PC(20) was significantly improved in the DCT group 0.51 +/−1.05 mg/ml versus 0.26 +/−0.84 mg/ml in the placebo group (p=0.034). The clinical and medication scores showed improvement in the DCT group (p=0.004). The results indicated that more stable airways were achieved with this add-on Chinese herb therapy.

In 2006, Chang et al. published the results of their study in Phytother Res 2006; 20:342-7 after comparing the therapeutic effects of the same 10 herbs but extracted in two different preparations. The clinical study was designed as a double-blind, placebo-controlled, randomized trial to observe the impact of the two herb preparations on allergic asthma. The two Chinese herbal formulae were coded as STA-1 and STA-2, both were the combination of Mai Men Dong Tang (MMDT) with 4 herbs and Lui-Wei-Di-Huang Wan (LWDHW) with 6 herbs. The difference between STA-1 and STA-2 was in the preparation procedure of the six herbs of LWDHW. These six herbs of LWDHW were milled to a powder in STA-1, but all the herbs in STA-2 were extracted by boiling water. In this study, 120 patients between the ages of 5 to 20 years with mild-to-moderate asthma were enrolled. These patients were treated with either STA-1 (44 patients) at a dose of 80 g/kg/day or STA-2 (40 patients) at a dose of 80 g/kg/day, or placebo (16 patients) administered twice daily for 6 months. Completion rate was 88%, 80% and 80% for STA1, STA2 and placebo respectively. The main outcome measures were a daily diary record of symptoms, supplementary bronchodilator and glucocorticoid treatment, changes of pulmonary function (forced expiratory volume in 1 s), changes of total and Dermatophagoides pteronyssinus (DP)-specific IgE and side effects. The results showed a statistically significant reduction of symptom scores, systemic steroid dos; total IgE and specific IgE levels in the STA-1 group as compared to the placebo. Furthermore, STA-1 improved pulmonary lung function (FEV1) compared with the placebo group. However, STA-2 treatment did not show significant improvement in any of above parameters. The authors speculated that certain compounds that have anti-inflammatory effect in LWDHW might be heat sensitive. However, chemical analysis would be required to support this hypothesis. This study also reported that there were no obvious adverse effects noted among the groups during the intervention period. These results suggested that STA-1 may be helpful for the treatment of mild-to-moderate chronic asthma. This same group also published an animal model study which demonstrated that STA-1 could effectively suppress the DP induced allergic reactions as evidenced by significantly reduced DP specific IgE. pulmonary inflammation and ABR.

The above scientific studies provided a strong foundation to support the combinational use of modern, western medicine and Chinese herbs, or the use of more natural herbal formulae, particularly when delivered directly into the lower respiratory tract, in order to achieve more desirable therapeutic effects without adding adverse side effects, Nonetheless, no report has been shown the use of Chinese herbs in a dry powder form for oral inhalation to treat asthma or other respiratory diseases. Embodiments of the invention disclosed herein also provide a new herbal formulation and an associated delivery mechanism by using a new PP-DPI for treating asthma and other respiratory tract diseases.

Many human diseases, such as diabetes or stroke, or disorders such as hypertension, depression or obesity, require a long-term treatment. The ideal therapy should be easy to administer, with reduced or no side effect, and the effect can be experienced quickly. Currently, many drugs are available but their dosage efficacy and side effects can be improved. PP-DPIs, in accordance with embodiments of the invention, further provide preferred means to deliver these chemical drugs. These drugs can be delivered directly to the patient's respiratory tract using a PP-DPI after a process which formulates each of these drugs into a fine dry powder form.

Biologics represent a new category of drugs and have rapidly gained momentum in recent years. Antibodies and their derivatives, particularly human monoclonal antibodies, are a rapidly growing category of targeted therapeutic agents. In addition, small interfering RNA, cytokines, enzymes, and a variety of peptide drugs have been studied. Rapid discoveries of new drug targets, more effective engineering processes, and knowledge on the behavior of biologics in the body resulted in an increased number of biologics being made, available on the market or in the late phases of clinical testing.

There are currently about 200 therapeutic proteins on the market, of which about 10% have been rationally designed with respect to their pharmacokinetics. For example, over 20 monoclonal antibodies have been approved by the US Food and Drug Administration and the European Medicines Agency and are currently the fastest growing category of targeted therapeutic agents and are expected to remain attractive in their potential for the discovery of new drugs and therapies.

As for all drug therapies, the efficient and targeted delivery of biologics to the desired site of action is highly desirable. However, due to their unique features, biologics represent a specific challenge in formulation development. Most often, the main strategies used in the product development of small molecular weight drugs cannot be readily transferred into the product development of biologics. Modified/improved strategies are needed to meet the specific challenges linked to protein and peptide drugs. In addition, specific challenges and opportunities in nonclinical safety testing of biologics need to be addressed and optimized.

Most of the products currently on the market are designed for the parenteral route of administration. For example, monoclonal antibody drug products have been on the market for over 20 years and are still administered in an acute care setting through intravenous infusion. Some of these products are designed for self-injection by patients, mostly as subcutaneous inⱼections. The efficacy of many biologics would be significantly enhanced if they were administered via alternative routes. For example. limitations exhibited by human growth hormone may be overcome by alternative routes to currently applied subcutaneous injections, which would not only increase the compliance and convenience to the patients but also assure the required dosing accuracy, frequently an issue with subcutaneous administration.

Respiratory tract-targeted delivery also offers a noninvasive alternative both for local and systemic conditions. It provides fast onset and favorable pharmacokinetics, avoiding the harsh conditions of the GI tract and first-pass metabolism. The success of aerosol-based delivery of proteins and peptides will be dependent on physiological factors (e.g., breathing pattern, alveolar macrophages, etc.) and specific properties of biologics (e.g., molecular weight, lipophilicity, etc.). The protective mucus layer covering the airway epithelium is an additional barrier to drug absorption. The optimal particle properties of aerosols, such as size, size distribution, surface characteristics, and drug load, are essential for successful protein-based therapies. Although the failure of Exubera® (short-acting insulin-loaded dry powder inhalers system; Pfizer, Inc., New York, N.Y., USA) slowed the development of delivery systems via the pulmonary route, several promising lines might overcome the current limitations of this route.

In response to the high occurrence of discontinuation of treatment due to noncompliance, several new delivery devices have been proposed such as prefilled syringes, manual injector pens, autoinjectors, and needle-free devices. Significant improvements have also been made in the field of micro/nanomechanical device-based drug delivery. These devices have shown promise in developing carriers with controlled physicochemical properties (especially size). These delivery devices particularly target the pediatric population of patients, and seem to provide the confirmed advantages of needle-based technologies. It is expected that more technological improvements will be made in this yet not fully explored area. In particular, devices for special populations of patients, such as children, elderly, and arthritic patients, need to become the focus of further development.

### BRIEF SUNMARY

Disclosed herein are various embodiments of safe and effective medical devices for administering many traditional and modern medicines for the treatment of many human diseases or disorders. Such embodiments include devices that broaden the range of applications of traditional and modern medicines. Herbal medicines have been used for thousands of years in China and in other countries, and now after a special preparation process, these natural drugs can be administered by using a hand-actuated positive pressure dry powder inhaler (PP-DPI), which is non-breath-actuated. Modern western medicines have a similar need to be delivered with a more suitable device for increased efficacy while decreasing their adverse effects. Drugs in a fine powder form can be delivered painlessly, easily, accurately and directly to the lungs through a positive pressure assisted inhalation; this offers many advantages as compared to orally ingesting a large amount of the same drug. Even greater advantages are achievable than injected drugs due to the non-invasiveness of the PP-DPI. Embodiments of the invention will not only reduce the potential side effects caused by allowing a large amount of the foreign chemical or biological materials to enter the human body, as a consequence of conventional delivery methods, it will also reduce the waste of these natural or synthetic resources. Embodiments of the invention also include kits for treating asthma, diabetes, obesity, depression, infection, allergy, pain, and many other diseases or disorders by using hand-actuated PP-DPIs, in accordance with embodiments of the invention, to deliver the right, amount of the drugs in the form of unique therapeutic powders directly into the lower respiratory tract. Thus, embodiments of the invention are applicable for the delivery of both traditional and modern medicines.

### BRIEF DESCRIPTION OF THE DRAWINGS

The accompanying drawings are included to provide a further understanding of the present disclosure and are incorporated in and constitute a part of this specification, illustrate embodiments of the present disclosure, and together with the description serve to explain^{,}the principles of the present disclosure. In the drawings:

FIG. 1 schematically shows an overall design of a hand-actuated positive pressure dry powder inhaler in accordance with an embodiment of the invention;

FIG. 2 schematically shows a preferred embodiment of various parts of a hand-actuated positive pressure dry powder inhaler in accordance with an embodiment of the invention;

FIG. 2.1 schematically shows a preferred embodiment of various parts in more detail of a hand-actuated positive pressure dry powder inhaler in accordance with an embodiment of the invention;

FIG. 3 depicts a preferred embodiment of various parts of an air-pump for a hand actuated positive pressure dry powder inhaler in accordance with an embodiment of the invention;

FIG. 4 schematically shows a preferred embodiment of various parts of an airflow adaptor for a hand-actuated positive pressure dry powder inhaler in accordance with an embodiment of the invention;

FIG. 5 schematically shows a preferred embodiment of the positive pressure supplier and controller of a hand-actuated positive pressure dry powder inhaler in accordance with an embodiment of the invention;

FIG. 6 schematically shows a preferred embodiment of the air pump and an airflow adaptor of a hand-actuated positive pressure dry powder inhaler in accordance with an embodiment of the invention;

FIG. 7 schematically shows a preferred embodiment of the air pump and a controller of a hand-actuated positive pressure dry powder inhaler in accordance with an embodiment of the invention;

FIG. 8 schematically shows a preferred embodiment of the air-pump and its controller of a hand-actuated positive pressure dry powder inhaler with a single dose loading capacity inhaler in accordance with an embodiment of the invention; and

FIG. 9 schematically shows a preferred embodiment of the air-pump and its controller of a hand-actuated positive pressure dry powder inhaler with multiple-doses loading capacity inhaler in accordance with an embodiment of the invention.

### DETAILED DESCRIPTION

The following description should be read with reference to the drawings, wherein like reference numerals indicate like elements throughout the several views. The drawings, which are not necessarily to scale, are not intended to limit the scope of the claimed invention. The detailed description and drawings illustrate exemplary embodiments of the claimed invention. Those skilled in the art will recognize that the various elements described and/or shown may be arranged in various combinations and configurations without departing from the scope of the disclosure. The detailed description and drawings illustrate example embodiments of the invention.

All numerical values herein are assumed to be modified by the term “about.” The disclosure of numerical ranges by endpoints includes all numbers subsumed within that range (e.g., 1 to 5 includes 1, 1.5, 2, 2.75, 3, 3.80, 4, and 5).

As used in this specification and the appended claims, the singular indefinite articles “a”, “an,” and the definite article “the” should be considered to include or otherwise cover both single and plural referents unless the content clearly dictates otherwise. In other words, these articles are applicable to one or more referents. As used in this specification and the appended claims, the term “or” is generally employed to include or otherwise cover “and/or” unless the content clearly dictates otherwise.

References in the specification to “an embodiment, ” “embodiments,” “such embodiments,” etc., indicate that the embodiment described may include a particular feature, structure, or characteristic, but every embodiment may not necessarily include the particular feature, structure, or characteristic. Moreover, such phrases do not necessarily refer to the same embodiment. Further, any particular feature, structure, or characteristic described in connection with a particular embodiment is intended to be applied, incorporated or substituted into other embodiments. whether or not explicitly described, unless clearly stated to the contrary.

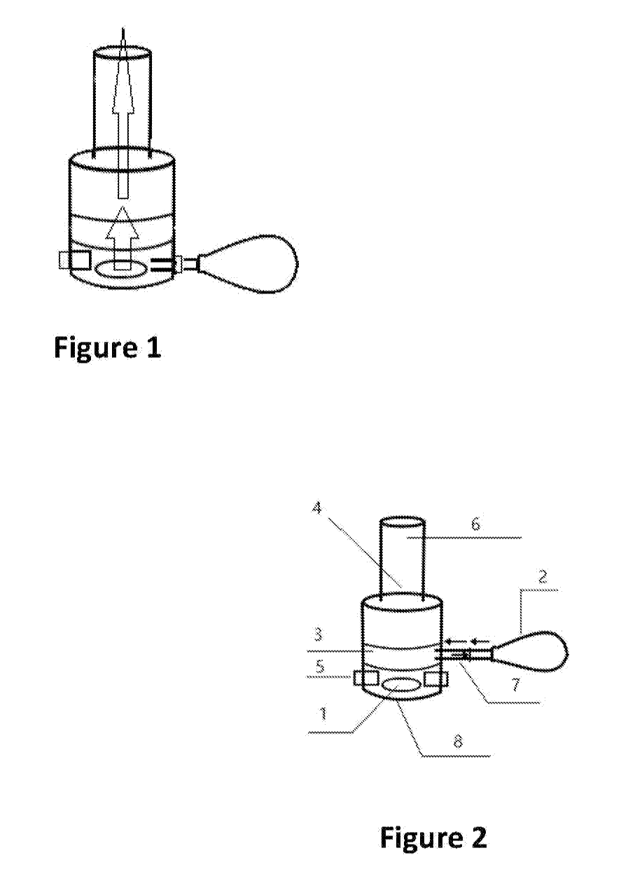

Referring to various embodiments of the invention disclosed herein, by integrating a hand-actuated positive pressure air-supplier (e.g. an air-pump), a dry powder inhaler (DPI) can be transformed into a positive pressure DPI (PP-DPI), or a non-breath-actuated inhaler, or a hand-actuated inhaler (FIG. 1). The PP-DPI is designed to deliver a single dose or multiple doses of the medicine, which is placed in an airflow chamber, trigger-released and carried with the hand-actuated airflow, passing through the air-passage inside of the inhaler, arriving to a mouthpiece, directly into the human respiratory tract.

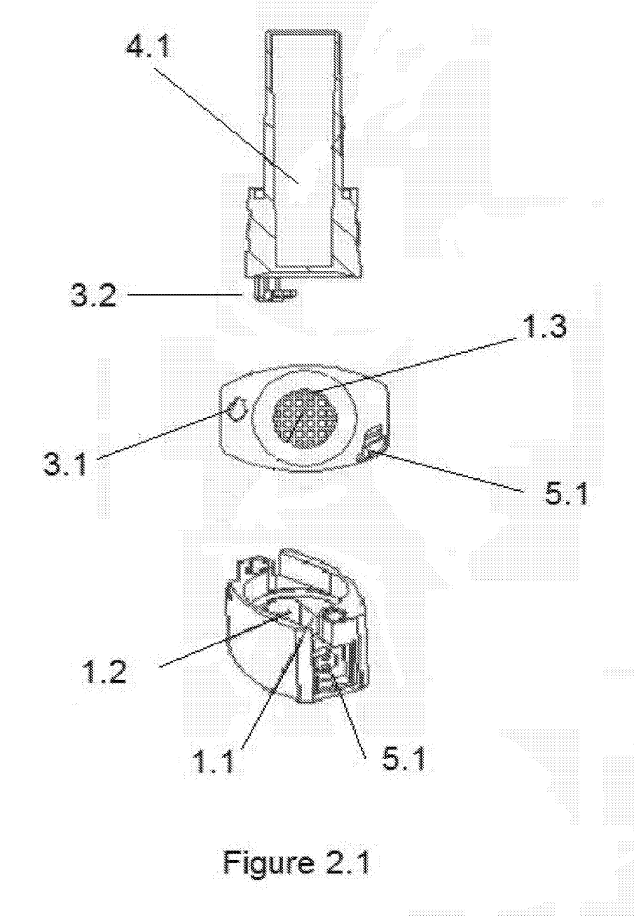

FIG. 2 shows a preferred embodiment of a PP-DPI which comprises: a) an airflow chamber 8 for the placement of a medicament 1 in a fine powder form, shown here contained in a pierceable capsule. The airflow chamber 8 forms the starting section of an air passage 4; b) a positive pressure supplier 2 with a one-way valve 7 connected to the airflow chamber 8; c) shutter 3 for closing the, airflow chamber 8; d) the air passage 4 may contain one or more vents (not shown); e) one or a group of piercing objects 5 placed in the airflow chamber 8 to pierce the medicament capsule 1 in order to release the medicament into the air passage 4; and f) a mouthpiece 6 as a part of the air passage.

FIG. 2.1 shows a preferred embodiment of a PP-DPI which further comprises: f) an air-inlet 5.1 to connect to the positive pressure supplier; g) curved internal wall-structure 1.1 to form a turbulence generating mechanism that generates turbulent air flow through the airflow chamber; h) the internal channel 1.2 formed by the wall-structure 1.1 for passing through the turbulent air flow to causes vibration of a medicament-holder in the air-flow chamber 8; i) the closure means or mechanism formed by parts 3.1 and 3.2; and j) the outlet air channel 4.1 which can be a part of the internal channel of the mouthpiece; k) the filter 1.3 to prevent any non-powder prices flow into the airway of a user.

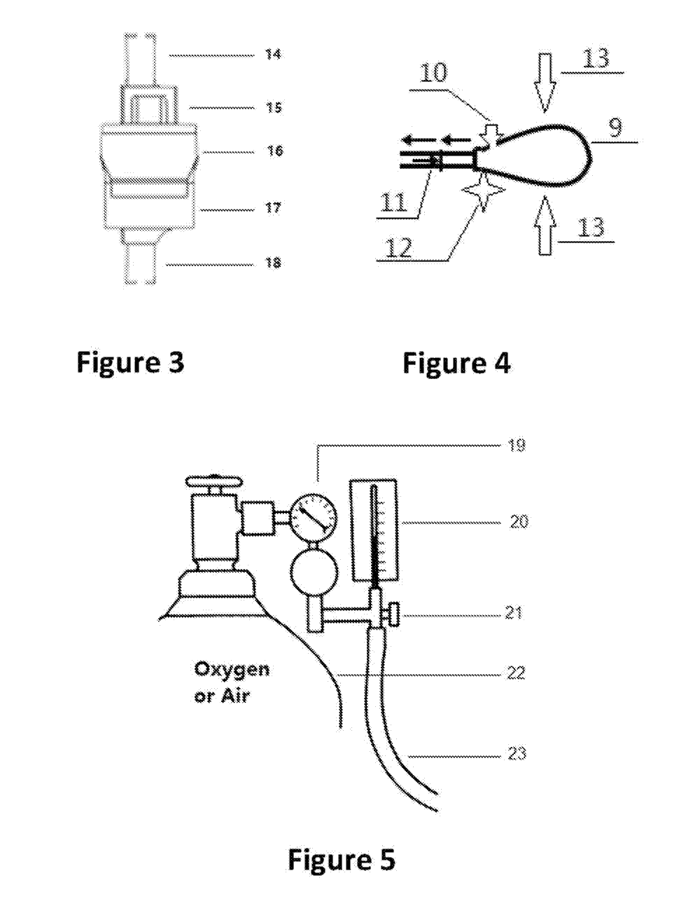

FIG. 3 depicts various parts of a preferred embodiment of an air-pump or a pneumatic pump for a hand-actuated PP-DPI. This is a small but powerful air pump to supply sufficient air pressure to the dry powder inhaler, and as such. the whole set is still portable. The air-pump 9 can be optionally equipped with a sensor 12 for, sensing air pressure before or during patient use. Depending on the different designs of the DPI, this adjustable air-pump can supply the right pressure to ensure the right airflow rate for actuating and releasing the powder, turn the powder into an aerosol, and bring the aerosol into the mouthpiece. A spring piston pump can also be used for this purpose. although an accurate adjustment mechanism would be more desirable. An air compressor can be used when the kinetic energy of the air is depressurized, with the air flowing into the DPI to trigger the release of the medicament in a powder form. The pump 9 represents an air supplier, either by a human hand to squeeze the bulb 13, or by electricity, plug in or battery, or from a container which already has compressed air, such as an air tank or oxygen tank. A switch 10, which is preferably hand activated, is use to turn the air supply on and off. This switch 10 also acts as a trigger to start the process of releasing the medicament 1 (in powder form) from the airflow/holding chamber 8 (shown in FIG. 2) and bring the powder via the, positive pressure airflow into the mouthpiece of the inhaler (shown in FIG. 2). An airflow controller or a valve 11 is preferably used to allow the compressed air to flow into the airflow chamber 8 of the dry powder inhaler, while preventing the dry powder from flowing out. A sensor and meter 12 is further used to indicate the air pressure under which the air is forced to flow into the airflow chamber 8.

FIG. 4 schematically shows various parts of a preferred embodiment of an airflow adaptor for a hand-actuated positive pressure dry powder inhaler. This airflow adaptor is small but has a one-size-fit-all design in order to connect the air supplier to the airflow chamber of a dry powder inhaler in any design. An air inlet 14 is preferably connected to the positive air-supplier 2 (shown in FIG. 2); An air outlet 18 is connected to the airflow chamber 8 (shown in FIG. 2) of the inhaler. Parts 15, 16 and 17 are the mechanical parts of the airflow adaptor. Through this airflow adaptor, the pumped air or compressed air flows into the airflow chamber of the inhaler (shown in FIG. 2) to actuate the release of the powder medicament.

FIG. 5 depicts a preferred embodiment of a positive pressure air supplier which can be used in conjunction with a hand actuated positive pressure dry powder inhaler. This compressed air supplier (such as an oxygen tank) with an air pressure meter 19 and pressure adjustment apparatus 20 can be readily used as the positive pressure supplier to any PP-DPI where the airflow adaptor (FIG. 4) is incorporated. The meter 19 indicates the air pressure level in the container (i.e. the pressure it supplies to the airflow chamber); while the 20 pressure adjustment apparatus indicates the rate at which the air flows into the inlet of the airflow chamber of a PP-DPI (shown in FIG. 2). The positive pressure air supplier also includes a switch 21, preferably is turned on and off by hand, which triggers the compressed air to flow into, the airflow chamber 8 of the PP-DPI (shown in FIG. 2) and releases the medicament powder into the mouthpiece of the PP-DPI. Compressed air may be supplied via a supplier 22, or alternatively air maybe supplied by an air-pump powered by electricity, man-power or other natural or mechanical force. The positive pressure air supplier also includes a tube or a passage 23 for transmitting air from the air supplier to the airflow adaptor, which is directly connected to the airflow chamber of the PP-PDI shown in FIG. 2.

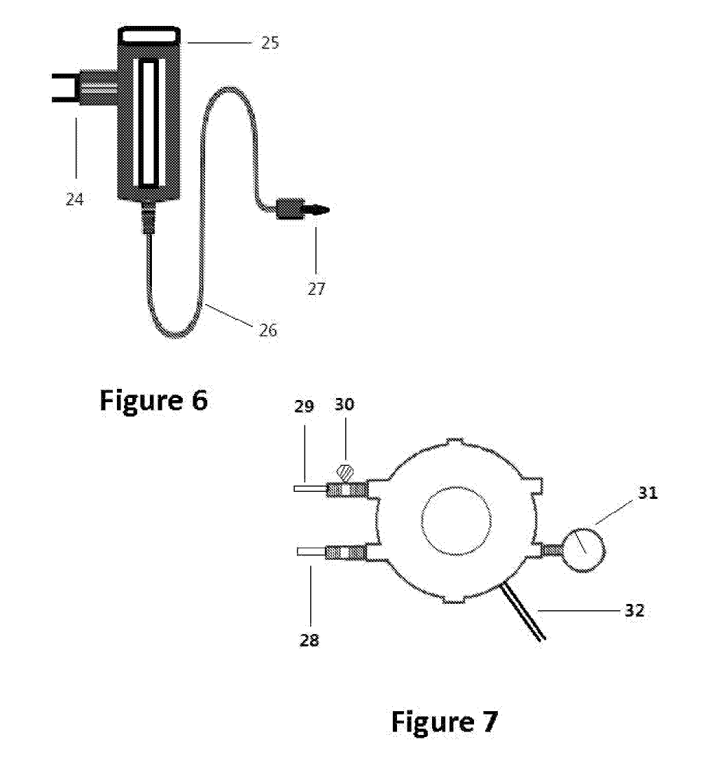

FIG. 6 depicts a preferred embodiment of a positive pressure air supplier and an airflow adaptor which can be used with a hand-actuated positive pressure dry powder inhaler. The air-pump 25 and an airflow adaptor 27 can be made as one set, or can be made as a kit to be used to supply the positive air-pressure to any dry powder inhaler. Also included in this preferred embodiment are an electricity plugin 24 or a battery or a group of batteries and an air tube 26 between the pressurized air supplier and an airflow adaptor. Using this combination of elements, a majority of currently existing DPIs, no matter their design, either a single dose or multiple doses DPI, can be converted into a PP-DPI.

FIG. 7 depicts another embodiment of an air-pump or a pneumatic pump for a hand-actuated PP-DPI. This is a small but powerful air pump which can supply sufficient air pressure to the dry powder inhaler, and as such, the whole set is still portable. The air-pump is preferably equipped with a sensor 31 for sensing and controlling the air pressure before or during the use. Depending on the different designs of the DPI, this adjustable air-pump can supply the needed pressure to ensure the right airflow rate to actuate and release the powder, change, the powder into an aerosol, and bring the aerosol into the mouthpiece. This air-pump is powered with electricity via an electrical supply 32. This embodiment also includes an air-inlet 28 of the air-pump, an air-outlet 29 of the air-pump, which connects to the inlet of the airflow chamber of the inhaler (shown in FIG. 2), and a switch (preferably a hand-switch) to turn on or off the air supplier. This switch 30 is a trigger to start the process of releasing the powder from its holding chamber 8 shown in FIG. 2 and bringing the medicament in the form of powder via the positive pressure airflow into the mouthpiece of the inhaler.

The hand-actuated PP-DPI can be implemented in a number of embodiments with either a single dose or multiple doses of the powder medicament.

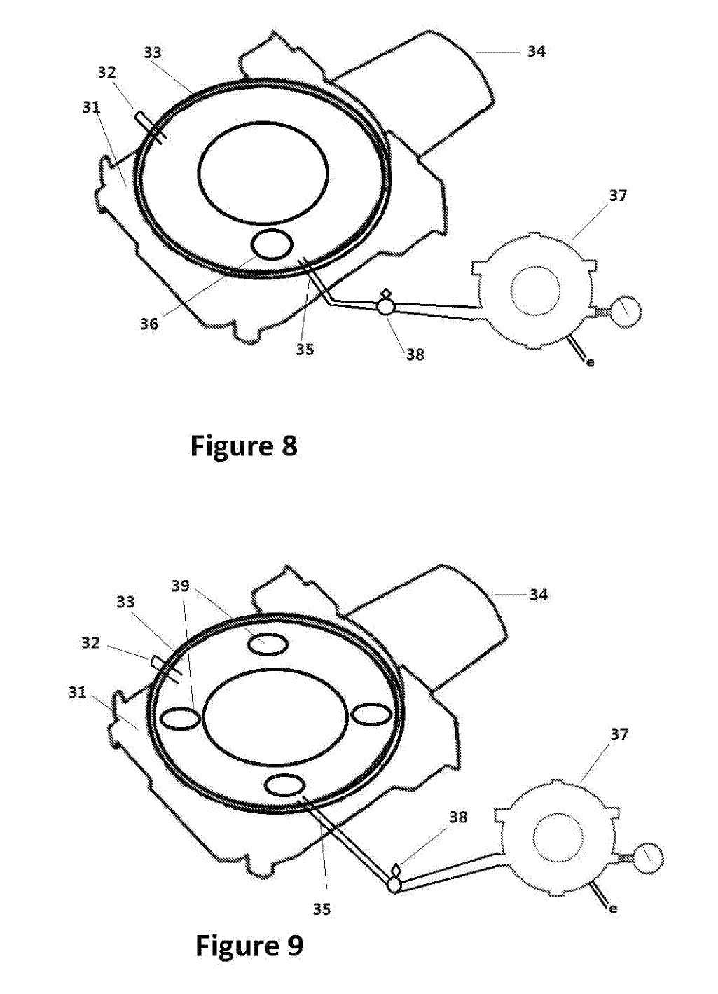

FIG. 8 depicts a preferred embodiment of an air-pump to supply a positive pressure to a hand-actuated PP-DPI with a single dose. One preferred embodiment comprises at least an airflow chamber, an air passage, a mouthpiece and an air supplier. The airflow chamber comprises: a holder 31 suitable for receiving a medicament capsule/wrap 36 which contains the medicament in a fine powder form and for forming the starting section of an air passage; a closure means or mechanism for closing the chamber, with the closure means or mechanism being moveable relative to the chamber body for closing the air passage through the chamber; a piercing object 32 suitable for piercing through the medicament capsule or wrap; a one-way air inlet 35 and a one-way air outlet 34 which together define>the air passage therebetween, the air passage comprising none, one or more vents (not shown); and a mechanism for generating turbulence in an air flow through the chamber 31 originated from an air-pump 37 such that, in use, the turbulent air flow causes vibration of the pierced medicament capsule/wrap 36 held by the chamber so as to assist in releasing the medicament contained within the pierced capsule/wrap 36 into the airflow, wherein the turbulence generated extends substantially the entire length of the chamber and the inhalation passage. The air supplier 37 provides positive pressure to the chamber to increase the turbulent air flow to enhance the release of medicament contained within the pierced capsule/wrap 36 into the air flow towards the mouthpiece 34. The operation makes it easier and more efficiently to administer the medicament to a patient.

The hand-actuated positive pressure dry powder inhaler can be implemented in another preferred embodiment as shown in FIG. 8 includes a one-way air inlet channel 35 through which air enters the inhaler; a chamber 31 that receives air from the inlet channel, the chamber containing a single actuator to which a powdered medicament 36 is attached, wherein the chamber 31 has a longitudinal axis, and wherein the inlet channel is generally co-axial with the longitudinal axis; a retaining member disposed at an end of the chamber opposite the inlet channel, the retaining member having one or more openings sized to permit air and the powdered medicament 36 to pass through the retaining member, and to prevent the actuator from passing through the retaining member; and an outlet channel through which air and the powdered medicament leave the inhaler through a mouthpiece 34, wherein the outlet channel is generally co-axial with the longitudinal axis of the chamber: wherein the geometry of the inhaler is such that a flow profile is generated within the chamber that causes the single actuator to repeatedly oscillate generally co-axially with the longitudinal axis, thus, by virtue of the repeated oscillations, dislodging the powdered medicament from the surface of the actuator to be entrained by the air and flow towards the mouthpiece 34; an air supplier 37 to provide positive pressure to the chamber to enhance the medicament flow towards the mouthpiece.

FIG. 9 depicts a preferred embodiment of an air-pump to supply a positive pressure to a hand-actuated PP-DPI with multiple doses. The hand-actuated positive pressure dry powder inhaler can be implemented in yet another preferred embodiment, which comprises a circulating airflow chamber 31 and an air passage connected by an integrally mounted hinge configured to enable the two parts of the inhaler to from an open position and a closed position; the inhaler further comprising a medicament dose 39 to be inhaled which is sealed in the at least part of the circulating airflow chamber 31 by a frangible membrane and being arranged such that the frangible membrane 39 is broken by an action of closing the inhaler prior to use and the dose is exposed to an entraining airflow 35 in the circulating airflow chamber upon a hand-triggered airflow and the patient's inhalation, and wherein the circulating airflow chamber 31 having air and entrained substance particles circulating within further comprises an axis and, along the axis, an one-way air inlet 35, a closed base and a vortex finder, the chamber being shaped such that at least, a part of the chamber decreases in cross-sectional area in a direction away from the one-way air inlet 35; wherein the one-way air inlet is configured to direct the air to circulate around the periphery of the chamber 33, and the vortex finder is configured such that the air exits therethrough after a reversal of axial direction at the base of the chamber to set up a reverse flow cyclone in the chamber wherein air circulates in, two generally concentric overlapping columns in opposite axial directions; and an air supplier 37 to provide positive pressure through the one-way air, inlet 35 to the circulating airflow chamber 31 to enhance the releasing of the medicament 39 contained within the frangible membrane and allowing it to flow easily towards the mouthpiece.

The hand-actuated positive pressure dry powder inhaler can be implemented in yet another preferred embodiment, which comprises: a chamber; a dose container disk 33 rotatably secured within the chamber 31, wherein the dose container disk 33 comprises opposing upper and lower primary surfaces, a first row of circumferentially spaced apart through apertures associated with dose containers 39 at a first radius and a second row of circumferentially spaced apart apertures associated with dose containers 39 at a second radius; a first flexible sealant residing over the apertures in the upper surface, and a second flexible sealant residing over the apertures in the lower surface, and wherein the dose containers 39 have dry powder therein; and a piercing mechanism 32 configured to serially alternate between rows to pierce through the sealants of a respective dose container in the first row, then pierce through the sealants of a respective dose container in the second row, wherein the piercing mechanism 32 comprises: a rotatable drum; and an elongate piercing member operably associated with the rotatable drum and capable of reciprocal movements between piercing and non-piercing positions; an air supplier 37 to provide a positive pressure to the chamber 31 to facilitate the dry powder flow towards the mouthpiece 34.

The positive pressure air supplier of the hand-actuated positive pressure dry powder inhaler can be an air pump which is a hand-held squeezable bulb with a one-way valve which permits the air flow into the chamber only (preventing the powder medicament from being sucked out of the chamber), as shown in FIG. 2. The second one-way valve in the bulb is to permit the air to enter the squeezable bulb and generate positive pressure repeatedly. Another embodiment of the air pump can be powered with a battery or plug-in electricity with an on/off switch. A third embodiment of the air pump can be powered with an air-pressure-filled elastic container from which the positive pressure can be released upon turning on the switch of the container, and the pressured air forcefully flows into the airflow chamber to bring the fine powder to flow through the air passage, so a patient can receive the drug through the mouthpiece.

The positive pressure supplier of the hand-actuated positive pressure dry powder inhaler is advantageous over an existing negative pressure dry powder inhaler which either holds a single dose or multiple doses of the medicament. With a positive pressure air supplier, the DPI becomes a positive pressure dry powder inhaler, which can be used to consistently and accurately administer a dry powder medicament to a patient. Because the positive pressure makes the dry powder drug flow easily and consistently, only a small portion of the force that drives the dry powder aerosol flow into the respiratory tract is derived from the patient's lungs; consequently the variations in patients' inspiration capacity becomes a practically negligible factor in impacting the accuracy of this dry powder delivery.

The hand-actuated PP-DPI is designed to match the normal respiration rate. The normal respiration rate for an adult at rest is 12 to 20 breaths per minute, and the medium rate is 16 breaths per minute. Also, it is known that the tidal volume is the lung volume representing the normal volume of air displaced between a normal inhalation and exhalation when no extra effort is applied. In a healthy young adult, the tidal volume is approximately 500 ml per inspiration (Beardsell 2009). A healthy human body will alter the minute volume in an attempt to maintain physiologic, homeostasis. A normal minute volume while resting is about 5-8 liters per minute in humans, which is about the total of 0.5 liter per each of the 16 breaths. During light activities the minute volume may be around 12 liters. Riding a bicycle increases the minute volume by a factor of 2 to 4 depending on the level of intensity involved. The minute volume during moderate exercise may be between about 40 and 60 liters per minute (Zuurbier 2009). For setting a standard of the air flow rate of all PP-DPIs, the air flow of one (1) liter during the two (2) seconds inhalation period is set by this invention. Depending on the resistance of the airflow in various implementations of DPIs, the air volume flowing into the DPI during the 2-second standard period can vary from 0.5 to 1.5 liters, or even within a broader range. which can be achieved by adjusting the setting of the air-pump. The fluidity of the dry powder inside of the DPI, or the easiness of the dry powder being inhaled by a patient, can be kept constant among different DPIs by adjusting the positive pressure of the applied air so the individual patients can have a consistent dosage of the inhaled drug no matter which portable or stationary PP-DPI is used.

Using a hand-actuated PP-DPI can enable the patient to inhale the powder drug without extra effort other than the normal inspiration. Therefore, the volume of air supplied to the chamber of the inhaler under the positive pressure is targeted to reach a standardized 500 ml per second inhalation. The 500 ml/second air flowing into the inhaler matches well with the patient's normal cycle of breathing, 500 ml per second inhalation. Therefore, this is to set a new pharmaceutical standard for any new PP-DPI that the pump power or air pressure and the inhaler must be matched to ensure 500 ml/second is to flow through the chamber of the PP-DPI. During the drug administration via inhalation, it is easy for a patient to continue the inhaling action for two seconds, then 1000 ml of air will be supplied to PP-DPI and all powder drug should be delivered into the lung. Without use of the embodiments of the invention disclosed herein, the patient would need to have a 30 to 120 liters per minute inhalation capacity in order to inhale the powder drug. Now the patient will only need a basic breath capacity, such as 5 liters per minute, to inhale and receive the dry powder drug into the lung. Therefore, the PP-DPI allows an effortless inhalation of the dry powder drug.

After standardizing the inhalation flow rate by using a hand-actuated PP-DPI, the dosage of the powder drug administration will basically not be influenced significantly by the variation in the patients' lung capacity. The flow rate of the dry powder inhaler can be standardized by setting up the air supply under a metered and controlled positive pressure, which is an important aspect of this invention.

Due to potential differences among many DPI implementations, the volume and rate of air supplied to the airflow chamber of the inhaler under the positive pressure is targeted to reach within a range of 10 to 5000 ml per second. Preferably, the range of air flow rate is within 100 to 1000 ml per second. More preferably, the optimal range of air flow rate is within 300 to 700 ml per second.

It is common to use liters/minute to measure the air flow rate for inhalation using a DPI. In this invention, the air flow rate of the air flowing into the new PP-DPI under a positive pressure can also be expressed in term of liters/minute. The positive air pressure supplied to the PP-DPI in a variety of designs should require an air flow rate in the range of 5 to 105 liters/minute. Preferably, the positive air pressure supplied to the PP-DPI will provide an air flow rate in the range of 20 to 90 liters/minute. More preferably, the positive air pressure supplied to the PP-DPI requires an air flow rate in the range of 30 to 60 liters/minute.

Air-pumps suitable for use in a PP-DPI implementation are widely available. These air pumps can be readily adapted to supply the positive pressure air to a PP-DPI. Electricity driven air pumps arc popular and well known. and can be easily controlled with a meter to indicate the supplied air flow rate. Embodiments of the invention disclosed herein provide an adaptor, to enable currently available electric air-pumps to become the new positive pressure supplier to a PP-DPI. The adaptor can connect the air-pump with any PP-DPI by a one-size-fit-all design and is preferably made with special elastic and/or hard materials. The tip of the adaptor is preferably soft and flexible which can be easily inserted into the air inlet of any DPI.

Hand-held air pumps can also, be adapted to supply air to a PP-DPI. The elasticity of the bulb and the volume of bulb will, significantly impact the supplied air volume to the PP-DPI. These variations can be standardized after a few tests and the manufacturer can select the right size of the bulb and adjust its elasticity in order to supply the right amount of positive pressure under normal use.

Between the airflow chamber and the air-supplier, an adaptor is provided. This adaptor allows the supply of positive air pressure from a variety of potential sources, such as pneumatic pump, air-pump, air compressor, compressed gas-container, piston-like air pump, etc. The adaptor can be designed as a unique one-size-fit-all implementation. The tip of the adaptor is re-shapeable, or soft. or flexible which can be easily inserted into the air inlet of any DPI. A hard tip can also be provided to perform this function. Any hand-held air pumps within an appropriate capacity range can be used to supply the air to any DPI through an adaptor. The adaptor can be an independent part or an integral part of the air-supplier.

It is known that many TCM therapies are available but the TCM formulae are rarely delivered using a MDI or a DPI directly into a patient's lungs or lower respiratory tract. Now these TCM formulae can be delivered in a fine powder formulation by using a PP-DR Embodiments of the invention, as disclosed herein, improve the efficacy of many TCM therapies which have a long-established successful use history. These TCM formulae are manufactured into a new dry powder form, packaged into pierceable capsules or wraps, and then delivered by using a NP-DPI or PP-DPI directly into the lower respiratory tract to treat respiratory diseases, such as acute bronchitis, acute respiratory distress syndrome (ARDS), asthma, acute or chronic bronchiolitis, bronchopulmonary dysplasia, chronic bronchitis, Chronic obstructive pulmonary disease (hereinafter “COPD”), cystic fibrosis, emphysema, hantavirus pulmonary syndrome, hypersensitivity pneumonitis, influenza, lung cancer, pneumonia, primary pulmonary hypertension, pulmonary arterial hypertension, pulmonary fibrosis, pulmonary vascular disease, respiratory syncytial virus infection, severe acute respiratory syndrome, sleep apnea. or tuberculosis. The formulation, with minor adjustments, can also be used to treat upper respiratory tract diseases, such as common cold, sinusitis, allergic rhinitis, stridor, tonsillitis, epiglottitis, whooping cough (Pertussis), or croup.

Furthermore, embodiments of the invention, as disclosed herein, enable the combination of two or more protective factors from. TCM or modern medicines into a new, formula where the new, combined, formulation is better than using a single factor for managing asthma and for treating other respiratory diseases. One example is the use of a DPI to directly deliver a dry powder containing both epinephrine and the extract of an herb in a very fine powder form. While epinephrine can dilate the lower respiratory tracts, the extract of Radix Scutellariae (Huang Qin) reduces inflammation of the lower respiratory tract. The combination of several active ingredients from the herbal extracts can be safer and more effective than the drug made from a single chemical compound, particularly for those patients who are unable to achieve control of their asthma on an inhaled steroid alone. These combinational herbal formulae are formulated as a mixed fine powder to be used for treating a human disease, such as asthma, by using a NP-DPI or PP-DPI. These components in the original formulae can be extracted, spray dried prior to being made into a fine powder, then inhaled via PP-DPI or NP-DPI into the lower respiratory tract to achieve a high therapeutic efficiency. Numerous herbs can be used for making the combinational formulae. Below are some of these herbs: Aloe vera (Lu Hui), Astragalus Root (Huang Qi), Codonopsis (Dang Shen), Belladonna alkaloids (Dian Qie Jian), Cordyceps militaris (Dong Chong Xia Cao), Cortex Mori Albae Radicis (Sang Bai Pi), egg shell, Flos Tussilaginis Farfarae (Kuan-Dong-Hua), Fructus Corni Officinalis (Shan Zhu Yu), Fructus Perilla Frutescens (Su Zi), Ginseng (Ren Shen), Glycyrrhizae (Gan Cao), Herba Ephedrae (Ma Huang), Jujube (Da Zao), Nonglutinous Rice (Geng Mi), Ophiopogonis (Mai Men Dong), Peppermint (Bo He), Pinelliae Preparatum (Zhi Ban Xia), Radix Dioscoreae Oppositae (Shan Yao), Radix Rehmanniae Preparata (Shu Di Huang), Radix Scutellariae (Huang Qin), Rhizoma Alismatis Orientalis (Ze Xie), Sclerotium Poriae Cocos (Fu Ling), Semen Pruni Armeniacae (Xing-Ren), Sophora flavescens ait (Ku Shen), Tripterygium wilfordii (Lei Gong Teng), or Tuber Pinellia (Ban Xia).

The formula used in TCM can be made, in the new dry fine powder form and delivered by using the PP-DPI, as shown and described with respect embodiments of the invention disclosed herein invention. Thousands of such TCM formulae can be made in the dry powder form, suitable for PP-DPI delivery. Below is an exemplary list of such TCMs which are suitable either alone or in combination with others for DPI delivery: An Zhong San (Calm the Middle Powder), Ba Wei Di uang Wan (Eight-Ingredient Pill with Rehmannia), Ba Zhen Tang (Eight-Treasure Decoction), Bai He Gu Jin Wan (Lilium Teapills), Ban Xia Bai. Zhu Tian Ma Tang (Pinellia, Atractylodes Macrocephala, and Gastrodia Decoction), Ban Xia Xie Xin Tang (Pinellia Decoction to Drain the Epigastrium), Bao Chan Wu You Fang (Preserve Pregnancy and Care-Free Decoction), Cang Er Zi (Upper Chamber Teapills), Chai Ge Jie Ji Tang (Bupleurum and Kudzu Decoction to Release the Muscle Layer), Chai Hu Gui Zhi Tang (Bupleurum and Cinnamon Twig Decoction), Chai Hu Qing Gan Tang (Bupleurum Decoction to Clear the Liver), Dang Gui Bu Xue Tang (Tangkuei Decoction to Tonify the Blood), Dang Gui Jing (Tang Kwei Gin), Dang Gui. Liu Huang Tang (Tangkuei and Six-Yellow Decoction), Dang Gui Nian Tong Tang (Tangkuei Decoction to Lift the Pain), Dang Gui Shao Yao San (Tangkuei and Peony Powder), Dun Sou San (Long-Bout Cough Powder), Er Zhu Tang (Two-Atractylodes Decoction), Gan Lu Yin (Sweet Dew Decoction), Ge Gen Huang Qin Huang Lian Tang (Kudzu, Coptis, and Scutellaria Decoction), Gua Lou Zhi Shi Tang (Trichosanthes Fruit and Immature Bitter Orange Decoction), Gui Qi Jian Zhong Tang (Tangkuei and Astragalus Decoction to Construct the Middle), Gui Zhi Tang (Cinnamon Twig Teapills), Hua San (Sophora Japonica Flower Powder), Huang Qi Jian Zhong Tang (Astragalus Decoction to Construct the Middle), Ling Gui Zhu Gan Tang (Poria, Cinnamon Twig, Atractylodes Macrocephala, and Licorice Decoction), Liu Wei Di Huang Wan (Six Flavor Teapills), Ma Huang Xing Ren Gan Cao Shi Gao Tang (Ephedra, Apricot Kernel, Licorice, and Gypsum Decoction), Mai Men Doug Tang (Ophiopogonis Decoction), Qiang Huo Sheng Shi Tang (Notopterygium Decoction to Overcome Dampness), Qing Bi Tang (Clear the Nose Decoction), Qing Fei Tang (Clear the Lung Decoction), Qing Hao I3ie Jia Tang (Artemisia Annua and Soft-Shelled Turtle Shell Decoction), Qing Shang Fang Feng Tang (Clear the Upper and Guard the Wind Decoction), Qing Shu Yi Qi Tang (Clear Summer-Heat and Augment the Qi Decoction), Qing Xin Li Ge Tang (Clear the Epigastrium and Benefit the Diaphragm Decoction), Qing Xin Lian Zi Yin (Lotus Seed Decoction to Clear the Heart), Qing Zao Jiu Fei Tang (Eliminate Dryness and Rescue the Lung Decoction), Ren Shen Yang Ying Tang (Ginseng Decoction to Nourish the Nutritive Qi), San Huang Xie Xin Tang (Three-Yellow Decoction to Sedate the Epigastrium), San Zhong Kui Jian Tang (Disperse the Swelling and Break the Hardness Decoction), Shao Yao Tang (Peony Decoction), Shi Liu Wei. Liu Qi Yin (Sixteen-Ingredient Decoction to Flow Qi), Shou Wu Zhi (Shou Wu Essence), Shu Jing Huo Xue Tang (Relax the Channels and Invigorate the Blood Decoction), Si Jun Zi Tang (Four Gentlemen Teapills), Si Ni San (Four Pillars Teapills), Si Wu Tang (Four Substances For Women), Tao He Cheng Qi Tang (Peach Pit Decoction to Order the Qi), Wei Ling Tang (Calm the Stomach and Poria Decoction), Wen Qing Yin (Warming and Clearing Decoction), Wu Zhu Yu Tang (Evodia Decoction), Xiang Sha Ping Wei San (Cyperus and Amomum Powder to Calm the Stomach), Xiao Chai Hu Tang (Minor Bupleurum Decoction), Xiao Cheng Qi Tang (Minor Order the Qi Decoction), Xie Huang San (Drain the Yellow Powder), Xin Yi Qing Fei Yin (Magnolia Decoction to Clear the Lung), Xue Fu Zhu Yu Tang (Stasis In The Mansion Of Blood Teapills), Yang Xin Tang (Nourish the Heart. Decoction), Yi Gan San (Restrain the Liver Powder), Yin Chen Hao Tang (Artemisia. Scoparia Decoction), Yin Qiao San (Honeysuckle and Forsythia Powder), Yu Ping Feng San (Jade Screen Teapills), Yu Nu Jian (Jade Woman Decoction), Zhe Chong Yin (Break the Conflict Decoction), Zhi Gan Cao (Honey-fried Liquorice Root), Zhi Sou San (Stop Coughing Powder), Zhu Ling Tang (Polyporus Decoction), Zhu Ye Shi Gao Tang (Bamboo Leaves and Gypsum Decoction), or Zi Yin Jiang Huo Tang (Nourish Yin, and Descend the Fire Decoction). It is noted that potentially suitable TCM formulae are not limited to the above list—numerous other TCM medicaments are also possible candidates.

Many drugs, used in modern medicine for specifically treating respiratory diseases have been delivered by using the best, available device during the research and development phase. If the chemical drug was delivered by using a NP-DPI, the same chemical drug can be delivered using the newly invented PP-DPI to reduce the dosage variation due to differences in the patients' inhalation capacity. If these chemical drugs were delivered via injection, now these same chemicals can also be potentially made into a dry powder form and delivered using a PP-DPI to eliminate the pain, cost and infection risk due to injection. If the drug was delivered though oral intake and then absorbed into blood circulation, these chemicals can also potentially be re-formulated into a fine powder form and delivered by using a PP-DPI to improve the efficacy and to reduce their side effects.

The first group of drugs currently being administered, using a breath-actuated NP-DPI for the treatment of respiratory diseases, can be delivered by using a hand-actuated PP-DPI, in accordance with an embodiment of the invention, to reduce the dosage variation. The drug ingredients are, but not limited to, albuterol, salmeterol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol, or (-)-4-amino-3,5-dichloro-alpha-[[[6-[2-(2-pyridinyl) ethoxy]hexyl]met-hyl]benzene-methanol. The drugs to be delivered in a powder form by using a PP-DPI can be in the form of salts, esters, etc., to thereby optimize the activity, efficacy and/or stability of the medicament.

A large group of the drugs currently used in modem medicine to treat a variety of diseases and/or disorders are delivered mainly through oral intake, injection, patch, spray and inhalation. Depending on the specific chemical and physical characteristics of the chemical or biological preparation, each drug had an optimal delivery route identified during the research and development stage of that drug. prior to the invention of the present PP-DPI. Broadly speaking, after overcoming these disadvantages of currently available non-positive pressure based dry powder inhalers, these drugs should be re-evaluated for their suitability for delivery using the, new PP-DPI device for potentially improved efficacy and side effects reduction. Even for certain historically failed drugs due to their poor delivery route in the past, they may be revived if that drug can be appropriately delivered by using the new PP-DPI device. For developing any new drug, the PP-DPI delivery device needs to be considered. In other words, the new PP-DPI device has, a potential to become the most commonly used medical device to deliver a variety of therapies due to its numerous advantages and capabilities.

It is beneficial to pharmaceutical industry and patients to consider using the PP-DPIs, in accordance with the embodiments of the invention, to deliver many medicines directly into the lungs for improving the therapeutic efficacy of these chemicals. Two basic requirements for selecting any chemical for delivery by using the PP-DPI are: (1) it is safe after being directly delivered into the lungs; (2) it is effective after entering into blood circulation though the lungs. Any drug meeting these two requirements can be delivered by using the newly invented PP-DPI. Some examples are (but not limited to): acetaminophen and/or propoxyphene for arthritis; aclidinium, budesonide, formoterol, roflumilast, umeclidinium, vilanterol for COPD; adalimumab, leflunomide, tocilizumab for rheumatoid arthritis; albuterol, montelukast, methylprednisolone, mometasone/ formoterol, or ipratropium for asthma; alinotriptan, diclofenac, eletriptan for migraine alprazolam, amitriptyline, bupropion, fluoxetine, vilazodone for depression; alprostadil, sildenafil, tadalafil, vardenafil, for erectile dysfunction; alteplase, aspirin, clopidogrel for stroke; amifostine, chlorambucil, cyclophosphamide, fluorouracil, or vincristine for cancer; amitriptyline for depression; amlodipine besylate for hypertension; amoxicillin, ceftriaxone, ciprofloxacin for urinary tract infection; amphetamine sulfate for attention deficit hyperactivity disorder; aripiprazole, lurasidone. risperidone, ziprasidone for schizophrenia;

atenolol for angina; atorvastatin calcium for osteoarthritis; atorvastatin, ezetimibe, pitavastatin. rosuvastatin, simvastatin for high cholesterol; azithromycin, levofloxacin, moxifloxacin for bronchitis; budesonide, sulfasalazine for inflammatory bowel disease; bupropion hydrochloride for depression; candesartan cilexetil for hypertension: carisoprodol for pain; ceftazidime for endocarditis: Celecoxib, Meloxicam, naproxen/esomeprazole for osteoarthritis; cetirizine, fluticasone furoate for hayfever; chlorpheniramine/acetaminophen, diphenhydrarnine got colds & Flu; clonazepam, lamotrigine, lurasidone for bipolar disorder; conjugated estrogens, zoledronic acid for osteoporosis; diethylpropion, lorcaserin, methamphetamine, phentermine for weight control; diphenhydramine for allergy; disopyramide. dofetilide for arrhythmia donepezil for Alzheimer's; duloxetine for depression; emtricitabine/rilpivirine/tenofovir for AIDS/HIV; enalapril, nebivolol, olmesartan, triamterene/hydrochlorothiazide for hypertension; epinephrine, epinephrine isomers, ephedrine for asthma and COPD; escitalopram for anxiety; esomeprazole for GERD (Heartburn); estazolam, flurazepam, olanzapine for insomnia; esterified. estrogens/methyltestosterone for menopause disorder; divalproex sodium ethosuximide, levetiracetam, primidone for seizures; ezetimibe for high cholesterol; fluoxetine for depressive disorders; gabapentin for nerve pain; heparin sodium for blood clots in the veins, arteries, or lungs; hepatitis b vaccine for hepatitis B; hydrochlorothiazide for edema; hydrocodone bitartrate for severe pain; hydromorphone. oxycodone, tapentadol for pain; hyoscyamine, mirabegron for urinary incontinence; ibuprofen for inflammation and pain; immune globulin for hepatitis A; insulin, insulin glargine, insulin lispro, pramlintide for diabetes (Type 1); insulin detemir, liraglutide, repaglinide, empagliflozin for diabetes (Type 2); isotretinoin for acne; levocetirizine for allergies; levofloxacin for bacterial infections; levothyroxine, liothyronine, thyroid for hypothyroidism; lisdexamfetamine for ADHD; loratadine for allergy; lorazepam for anxiety disorders; morphine, oxycodone for moderate to severe pain; naproxen for arthritis pain or inflammation; nifedipine for hypertrophic cardiomyopathy; oseltamivir, zanamivir for influenza; paroxetine hydrochloride for depression; pegloticase for gout; penicillin for bacterial infection; phentermine for obesity; pregabalin for fibromyalgia; pregabalin for seizures; quetiapine fumarate for schizophrenia, ranolazine for angina; risperidone for schizophrenia and symptoms of bipolar disorder; sertraline hydrochloride for depression; sildenafil citrate for pulmonary arterial hypertension; tramadol hydrochloride for pain; valsartan for hypertension; vardenafil hydrochloride for impotence; venlafaxine hydrochloride for depression or zolpidem tartrate for sleep problems. The pharmaceutically active agent may be used in the form of salts, esters or solvates to thereby optimize the activity, efficacy and/or stability of the medicament.

Each ingredient has its required dosage for safety and effectiveness when it is delivered by using a PP-DPI. The dose of each of these current drugs in the marketplace or in the research and development phases was or will be established and these doses are served as references for deciding the dose in the PP-DPI formulation. In a broad range, the dosage of each of these drug substances used in, the PP-DPI formulation is expected to vary between 5 times higher to 100 times lower than its previously established dose using its established delivery route. The dry powder formulation may comprise about 50,00% to about 99.99% (w/w) of a carrier, such as inhalable lactose.

The dosage range of each of these modern drugs in a PP-DPI formulation is between 5 times higher and 10 times, lower than its actual amount in, an injection formation. Preferably, the same amount of the actual drug substance contains in a dose of the PP-DPI formulation as in its corresponding injection formulation.

The dosage range of each of these modern drugs in, a PP-DPI formulation is between 1.1 times higher and 100 times lower than its actual amount in an oral intake formation. Preferably, at least 10-times less amount of the actual drug substance is contained in a dose of the PP-DPI formulation than that in its corresponding oral formulation. More preferably, at least 2-times less amount of the actual drug substance is contained in a dose of the PP-DPI formulation than that in its corresponding oral formulation.

The dosage range of each of these modern drugs in a PP-DPI formulation is between 1.1 times higher and 2 times lower than its actual amount in a patch formation. Preferably, a 0.5-times less amount of the actual known drug substance is contained in a dose of the PP-DPI formulation than, that in its corresponding patch formulation.

The dosage, range of each of these modern drugs in a PP-DPI formulation is between 5 times higher and 5 times lower than its actual amount in a spray formation. Preferably, a 0.5-times less amount of the actual drug substance is contained in a dose of the PP-DPI formulation than that in its corresponding spray formulation.

The dosage range of each of these modern drugs in a PP-DPI formulation is between 1.1 times higher and 5.0 times lower than its actual amount in a known inhalation formation. Preferably, the same amount of the actual drug substance contains in a dose of the PP-DPI formulation as in its known inhalation formulation.

In one embodiment, the dosage range of inhalable epinephrine in a PP-DPI formulation is between 1.1 times higher and 2.0 times lower than its actual amount in the inhalation formation, which are 0.10 mg and 0.25 mg per inhalation. Due to variations in the pharmaceutical industry and a patient's body size and age, the dry powder formation of the inhalable epinephrine for PP-DPI formulation comprises approximately 0.01 mg to about 2.00 mg of epinephrine or its equivalent compounds; or alternatively about 0.05 mg to about 1.00 mg; or alternatively about 0.10 mg to about 0.50 mg; or alternatively about 0.15 mg to about 0.30 mg; or alternatively about 0.20 mg to about 0.25 mg; or alternatively about 0.22 mg to about 0.25 mg. The dry powder formulation may comprise about 95.00% to about 99.99% (w/w) of a carrier, such as inhalable lactose.

The third and a new group of therapies are biologics, or so called big molecules. These biologics are not easy or suitable for oral intake as many factors in the gastrointestinal tract will denature these big molecules. With the exception of certain big molecules which could cause toxicity if inhaled into the lungs, such as Abobotulinum Toxin Type A or Incobotulinumtoxin A, most biologics currently administered by injection or inhalation can be delivered by using the newly invented PP-DPI. In other words, PP-DPI delivery can be used to replace most injection form of the drugs to eliminate a number of drawbacks of the injectables—these include: difficulty in manufacturing the stable dose; difficulty to keep a long shelf-life; difficulty in storage before, use and during transportation (keep in refrigerator or a freezer, for example); pain and the risk of potential infection during injection, and the high cost and inconvenience caused by the need to perform the injection by a medical professional. These biologics would undergo a freeze-drying or dry spray process before being packed into a dry powder dosage for administering with a PP-DPI. A dry powder is more stable than an injection liquid. Disadvantages associated with injectables can be largely eliminated when these biologics are administered with a PP-DPI. Numerous biologics are available and many of them can be made into a dry powder formulation, which then can be administered by using the newly invented PP-DPI. Without limitation, these common drugs are listed here as a part of the embodiments of the invention as candidates for delivery using the newly invented PP-DPI: 90Y-Ibritimomab tiuxetan, Abatacept, Abciximab, Adalimumab, ado-trastuzumab emtansine, Aflibercept, Agalsidase beta, Albiglutide, Aldesleukin, Alefacept, Alemtuzumab, Alemtuzumab, Alglucosidase alfa, Afteplase, Anakinra, asparaginase Erwinia chrysanthemi, Basiliximab, Becaplermin, Belatacept, Belimumab, Bevacizumab, Bortezotnib, Brentuximab vedotin, Canakinumab, Capromab, Pendetide, Certolizumab pegol, Cetuximab, Collagenase, Collagenase Clostridium Histolyticum, Daclizumab, Darbepoetin alfa, Dasatinib, Denileukin diftitox, Denosumab, Domase alfa, Drotrecogin alfa, Dulaglutide, Ecallantide, Eculizumab, Efalizumab, Elosulfase alfa, Epoetin alfa, Erlotinib, Etanercept, Everolimus, Fanolesomab, Filgrastim, Galsulfase, Gefitinib, Gemtuzumab, Glucarpidase, Golimumab, Ibritumomab tiuxetan, Idursulfase, Imatinib, Infliximab, Interferon alfa-2a, Interferon alfa-2b, Interferon alfa-2b, Interferon alfacon-1, Interferon alfa-n3, Interferon alpha, Interferon beta-1a, Interferon Beta-1b, Interferon gamma-1b, Interleukin-2, Ipilimumab, Lapatinib, Laronidase, Lenalidomide, Methoxypolyethylene glycol epoetin beta, Metreleptin, Muromonab-CD3, Natalizumab, Nofetumomab, Obinutuzumab, Ocriplasmin, Ofatumumab, Omalizumab, Oprelvekin, Palifermin, Palivizumab, Palivizumab, Panitumumab, Pegaspargase, Pegfilgrastim, Peginterferon alfa-2a, Peginterferon alfa-2b, Peginterferon beta-1a, Pegloticase, Pembrolizumab, Pertuzumab, Ramucirumab, Ranibizumab, Rasburicase, Raxibacumab, Reteplase, Rilonacept, Rituximab, Sargramostim, Siltuximab, Sorafenib, Sunitinib, tbo-filgrastim, Tenecteplase, Thalidomide, Tocilizumab, Tositumomab, Trastuzumab, Ustekinumab, Vedolizumab, Vermurafenib, ziv-aflibercept.

The dosage range of each of these biologics in a PP-DPI formulation is between 5.0 times higher and 5 times lower than its actual amount in the currently-known injection formulation. Preferably, the same amount of the actual biologic substance is contained in a dose of the PP-DPI formulation as in its corresponding injection formulation.

The dosage range of each of these biologics in a PP-DPI formulation is between 2.0 times higher and 2 times lower than its actual amount in the currently-known inhalation formulation. Preferably, the same amount of the actual biologic substance is contained in a dose of the PP-DPI formulation as in its corresponding known inhalation formulation.

The dry powder inhalation by using a PP-DPI offers a broad range of new applications. Although many existing dry powder inhalers have been used for many years to deliver one or two chemical drugs, the new PP-DPI can replace the NP-DPI for a much better performance. PP-DPI can also be used to deliver a mix of multiple agents to improve the therapeutic effect. In addition, using a PP-DPI can deliver the new combination of the chemical drug and the extract from one or more herbal medicines. A number of ingredients can be combined, micronized, formulated, mixed, homogenized, packed (encapsulated) and then loaded into a PP-DPI for inhalation. Some of these ingredients are bronchodilator, antimicrobial agent, anti-inflammatory, agent, anti-allergic agent, antihistamine agent, immunomodulation agent, and tissue healing agents. Many fine extracts of the formulated. TCM herbs can be used in combination. Using an appropriate process, these therapeutic agents can be incorporated into the dry powder and delivered into the lower respiratory tract with a PP-DPI. Different formulations with the correctly selected active ingredients can be made to meet the different needs for different patients with different diseases.

Embodiments of the invention, as disclosed herein, provide a number of new therapies offered through the combinational use of a medical device—a dry powder inhaler and correctly selected active therapeutic ingredients. It is known that developing a new formulation delivered with the metered dose inhaler (MDI) has a number of, technical challenges particularly due to the fact that the propellant may not be safe and preservative agents are needed to maintain a required long shelf-life of the liquid drug. Formulation of the powder delivered with a DPI in general is easier than that delivered with a MDI. Due to the reduced need for using other components, the formulated drug in the dry powder form is much purer than the drug in the liquid form. When it is appropriate, the pure active ingredient(s) alone can be delivered with a PP-DPI. In practice, any drugs delivered using a MDI now can be delivered by using a PP-DPI since the need for a strong inhalation capacity for using a breath-actuated NP-DPI no longer exists with the hand-actuated PP-DPI.

Embodiments of the invention, as disclosed herein, are also particularly suited for a patient who is suffering from a hard to treat asthma that is not responsive to the common therapy or the standard of care treatment. The new combination of several protective active ingredients will improve the response rate. Administering to the patient an effective amount of the combinational ingredients directly to the lungs through a PP-DPI can quickly result in the symptom relief.

Preferably, the active ingredient(s) is administered by using a PP-DPI for the immediate relief of asthma symptoms or acute allergic reaction, such as anaphylaxis. The patient is not required to have a strong inhalation capacity in using a PP-DPI, which is critically important for rescuing the patient when a severe allergic reaction occurs.

Embodiments of the invention, as disclosed herein further include methods for treating respiratory diseases by administering an effective amount of the active ingredient in a fine powder form to a patient in need of such a treatment where the respiratory diseases or one or more manifestations of the respiratory diseases are non-responsive or substantially non-responsive to treatment with the current standard of care. Additionally, embodiments of the invention, as disclosed herein, provide methods of treating a severe and long-lasting episode of asthma by administering an effective amount of the active ingredient to a patient in need of such a treatment, where the severe and long-lasting episode of asthma or one or more manifestations of it are non-responsive or substantially non-responsive to treatment with the standard of care. According to one embodiment of the invention, the active ingredient administered is an herbal formula alone or in combination with a chemical drug, with or without using a pharmaceutically acceptable carrier.

For example and without limitation, patients suffering from respiratory diseases (for example, those admitted to an emergency room because of an acute exacerbation of asthma), who are not responsive to the standard of care, can be treated with a combinational herbal formula and modern medicine (in addition to having been treated with the standard of care) and their treatment outcomes could be more favorable.

The particle size of any dry powder has a significant impact on the therapeutic result due to its distribution in the respiratory tract. For delivery using a PP-DPI, the particle size of the active ingredient(s) should be within 2-5 microns, It is well known in the pharmaceutical industry that the powdered medicament particles suitable for delivery to the bronchial or alveolar region of the lungs have an aerodynamic diameter of less than 10 micrometers, preferably within the range of 2 to 5 micrometers. Particles of powdered medicamnent and/or excipient may be produced by conventional techniques, for example by spray drying micronization, milling or sieving.

Other sized particles may be used if delivery to other parts of the respiratory tract is desired, such as the nasal cavity, mouth or throat. However, for treating any disease or disorder systemically, better results will generally result from pulmonary delivery so the therapeutics can enter into the blood circulation more quickly. The medicament may be delivered as a pure drug, or more appropriately, it is preferred that medicaments are delivered together with excipients (carriers) which are suitable for inhalation. Suitable excipients include organic, excipients such as polysaccharides (e.g. starch, cellulose and the like), lactose, glucose, mannitol, amino acids. and maltodextrins, and inorganic excipients such as calcium carbonate or sodium chloride. Lactose is a commonly used and preferred excipient. Additionally, medicament and/or excipient powders may be engineered with particular densities, size ranges, or characteristics. Particles may comprise active agents, surfactants, wall forming materials, or other components considered desirable by those of ordinary skill in the art.

Patients who have normal lungs will experience an ease in breathing with a powder formulation for a quick therapeutic effect, even when using a breath-actuated NP-DPI. After the accuracy of drug dose delivery is ensured by using the new PP-DPI, more patients will prefer this better and more user-friendly device in order to receive the medicine they need into their lungs. As is well known, the inhaled medicine works faster than the same medicine in a pill. In addition, the patients will likely need a smaller amount of medicine than they would orally, hence, side effects caused by excessive drug substances can be reduced or even eliminated due to the smaller exposure to the given drug. Thus, regarding potential side effects, PP-DPI delivery is expected to be advantageous than swallowing the pill.

PP-DPIs, in accordance with invention embodiments disclosed herein, are directed to a medical device, delivering significant improvements in aerosol delivery including better standardization of delivery, force, device function and patient use, greater reliability, more accurate dosing, and reduction of drug loss and potential adverse effects.

It is easy for the majority of adults and children 4 years and older either alone or under the supervision of an adult to use a hand-actuated PP-DPI. Below is the method or procedure of using a natural therapeutic kit containing a hand-actuated positive drive powder inhaler (PP-DPI) and an inhalable therapeutic natural powder, or using a modern therapeutic kit containing a hand-actuated PP-DPI and an inhalable pharmaceutically active powder, or using a therapeutic biologic kit containing a hand-actuated PP-DPI and an inhalable pharmaceutically active, biologic powder, or using a therapeutic herb kit containing a hand-actuated PP-DPI and an inhalable herb powder, or using a combinational therapeutic kit containing a hand-actuated PP-DPI and an inhalable powder of the combinational mix:

Step 1. Load or make sure the inhalable medicament is loaded in the airflow chamber of the hand actuated PP-DPI;
Step 2. Pierce the wrap or capsule containing the medicament;
Step 3. Place the mouthpiece inside the mouth after a deep exhalation;
Step 4. Use hand to turn on the supplier of the positive pressure to the airflow chamber to trigger the release of the medicament;
Step 5. Start a gentle inhalation at the same time of turning on the positive pressure supplier;
Step 6. After a gentle exhalation, repeat one more times of applying the positive pressure and inhaling the medicament; and
Step 7. Rinse mouth and clean the mouthpiece for future use.

### EXAMPLES

The following examples are provided to illustrate certain aspects of embodiments of the invention, and to aid those of ordinary skill in the art in practicing embodiments the invention. These examples are not to be construed as limiting the scope of the invention as these, and other equivalent embodiments will be apparent in view of the present disclosure, figures, and accompanying claims.

### Example 1: A Natural Herb Kit for Treating Rheumatoid Arthritis

A preferred embodiment of the kit contains a dry powder inhaler (DPI) and 100 capsules made by packing the dry powder extracted from Tripterygium wilfordii, a traditional Chinese herbal medicine. The herb, Tripterygium wilfordii, is completely dried, broken down to very tiny pieces, placed into water, heated to the boiling point, for 20 minutes and then kept overnight. The solid waste is removed and the extract is processed by spray drying to obtain the fine powder in the size of 2 to 10 micron. The micronized powder of 100 mg is then mixed with 100 mg inhalable lactose and homogenized. The mix in the total weight of 200 mg is filled into number 3 capsules. This kit, packed with an appropriate amount of the capsules along with a DPI, can be used by a patient with rheumatoid arthritis.

### Example 2: A Natural Therapeutic Kit for Treating Asthma or Improve Immunity

A preferred embodiment of the kit contains a DPI and an inhalable therapeutic natural powder made from mixing and homogenizing the extract in, powder form of six herbs for making the commonly used Chinese medicine Lui-Wei-Di-Huang Wan (LWDHW). Forty milligrams of each fine powder of the extract of Cortex Moutan Radicis (Paeonia Suffruticosa) (Mu Dan Pi), Fructus Corni Officinalis (Shan Zhu Yu), Radix Rehmanniae Preparata (Shu Di Huang), Rhizoma Alismatis Officnalis (Ze Xie), Rhizoma Dioscoreae Oppositae (Shan Yao), and Sclerotium Poriae Cocos (Fu Ling) are mixed and homogenized, milled into a very fine powder. The total 240 mg mix is then filled into number 3 capsules. This kit, packed with an appropriate amount of the capsules along with a DPI, can be used by a patient with asthma or by someone who needs to improve immunity.

### Example 3: A Therapeutic Powder Formulation for Diabetes

A preferred embodiment of the therapeutic powder formulation for diabetes is made by combining the fine powder of the extract (25 mg each) from adenophora, anemarrhena, asparagus root, dendrobium, glehnia, gypsum, lycium bark, ophiopogon, pueraria, raw rhmannia, scrophularia, trichosanthes root, and yu-chu. The mix is homogenized and packed in capsules for pulmonary administration using a PP-DPI.

### Example 4: A Therapeutic Powder Formulation for Digestive Disorder

A preferred embodiment of the therapeutic powder formulation for digestive disorder is made by combining the fine powder of the extract (40 mg each) from astragalus, atractylodes, dioscorea, ginseng, polygonatum, and pseudostellaria. The mix is homogenized and packed in capsules for pulmonary administration using a PP-DPI.

### Example 5: A Therapeutic Powder Formulation for Cleansing Toxicant in the Liver and Kidneys

A preferred embodiment of the therapeutic powder formulation for cleansing toxicant in the liver and kidneys is made by combining the fine powder of the extract (30 mg each) from aconite, alistna, cinnamon, cornus, epimedium, ho-shou-wu, and lyciutn fruit. The mix is homogenized and packed in capsules for pulmonary administration using a PP-DPI.

### Example 6: A Therapeutic Powder Formulation for Treating Abnormal Blood Triglyceride

A preferred embodiment of the therapeutic powder formulation for treating abnormal blood triglyceride is made by combining and mixing the fine powder of the extract (50 mg each) from astragalus, polygonatum, pseudostellaria, rehmannia, and trichosanthes root. The mix is homogenized and packed in capsules for pulmonary administration using a PP-DPI.

### Example 7: A therapeutic Powder Formulation for Improving Peripheral Blood Circulation

A preferred embodiment of the therapeutic powder formulation for improving peripheral blood circulation is made by combining and mixing the fine powder of the extract (25 mg each) from anemarrhena, astragalus, atractylodes (cangzhu), carthamus, codonopsis, gypsum, persica, rehtnannia, salvia, and tang-kuei. The mix is homogenized and packed in number 3 capsules for pulmonary administration using a PP-DPI.

### Example 8: A Therapeutic Kit for Relieving Respiratory Symptoms

A preferred embodiment of the therapeutic kit for relieving respiratory symptoms is made by packing a dry powder inhaler and 20 capsules of epinephrine with net weight of 0.22 mg each. Epinephrine is mixed with an inhalable lactose as a carrier. The mix is homogenized and packed in capsules for pulmonary administration using a PP-DPI. This therapeutic kit is packed with an appropriate amount of the capsules along with a PP-DPI.

### Example 9: A Combinational Therapeutic Kit for Relieving Respiratory Symptoms

A preferred embodiment of the combinational therapeutic kit for relieving respiratory symptoms is made by packing a dry powder inhaler and 20 capsules of epinephrine with net weight of 0.22 mg each. along with an extract of licorice (250 mg). Epinephrine is mixed with the fine powder of licorice extract. The mix is homogenized and packed in capsules for pulmonary administration using a PP-DPI. This therapeutic kit is packed with an appropriate amount of the capsules along with a DPI. It was easy for the patients, to inhale the drug to reduce asthma symptoms.

### Example 10: A Combinational Therapeutic Powder Formulation for Balancing Blood Sugar Concentration

A combinational therapeutic powder formulation for balancing blood sugar concentration is made by combining and mixing the fine powder of the extract (25 mg each) from alisma, atractylodes, coptis, ginseng, ho-shou-wu, lonicera, lycium bark, phellodendron, polygonatum, rehmannia, scrophularia, and yu-chu, alone with epinephrine (0.25 mg). The mix is homogenized and packed in capsules, for pulmonary administration using a PP-DPI.

### Example 11: A Therapeutic kit for Type 1 Diabetes

A preferred embodiment of the therapeutic kit for type 1 diabetes contains a hand-actuated positive drive powder inhaler (PP-DPI) and an inhalable insulin in the fine powder in the amount of 8 units per capsule. Insulin is mixed with lactose as a carrier. This is for oral inhalation using a PP-DPI at each meal to balance blood glucose level. The 8 units inhaled insulin replaced 8 units of subcutaneous injection of insulin by the patient at the beginning of the mealtime.

### Example 12: A Therapeutic Kit for Type 2 Diabetes

A preferred embodiment of the therapeutic kit for type 2 diabetes contains a hand-actuated positive drive powder inhaler (PP-DPI) and an inhalable insulin in the fine powder form in the amount of 4 units per capsule. Insulin is mixed with lactose as a carrier. The 4 units inhaled insulin replaces 4 units of subcutaneous injection of insulin at the beginning of the mealtime. It was easy for the patients to inhale the drug to achieve the purpose of blood glucose control.

### Example 13: A Therapeutic Kit for Influenza and Respiratory Tract Infection

A preferred embodiment of the therapeutic kit for influenza and respiratory tract infection contains a drive powder inhaler and an inhalable mixture of Shuang Huang Lian, a traditional Chinese medicine. This formula comprises three herbs: lonicera, scute and forsythia. This TCM formula is traditionally known as an antiviral, and is most commonly used for the treatment of respiratory infections, including upper and lower respiratory tract infections and acute bronchiolitis. The mix of the three extracts (each 100 mg) in the very fine powder is homogenized and packed in capsules for pulmonary administration using a PP-DPI. This therapeutic kit is packed with an appropriate amount of the capsules along with a DPI.

### Example 14: A Therapeutic Kit for Relieving Respiratory Symptoms (Asthma or COPD)

A preferred embodiment of the therapeutic kit for relieving respiratory symptoms (asthma or COPD) is made by packing a hand-actuated positive pressure dry powder inhaler and 20 capsules of albuterol with net weight of 0.20 mg each, along with inhalable lactose as a carrier. Albuterol is mixed with the fine powder of inhalable lactose with or without licorice extract. The niix is homogenized and packed in capsules for pulmonary administration using a PP-DPI. This therapeutic kit is packed with an appropriate amount of the capsules along with a DPI.

### Example 15: A Risk-Reduction Kit for Preventing Symptoms of Bronchospasm Prophylaxis

A preferred embodiment of the risk-reduction kit for preventing symptoms of bronchospasm prophylaxis contains a hand-actuated positive pressure dry powder inhaler and 20 capsules of albuterol with net weight of 0.22 mg each capsule, along with inhalable lactose as a carrier. Albuterol is mixed with the fine powder of inhalable lactose. The mix is homogenized and packed in capsules for pulmonary administration using a PP-DPI. This therapeutic kit is packed with an appropriate amount of the capsules along with a DPI. The user should administer the dry powder 15 minutes before exercise.

In accordance with the foregoing, embodiments of the invention can be further defined per the following:

1. A hand-actuated positive pressure dry powder inhaler (PP-DPI) for administering an inhalable medicament, comprising: an airflow chamber for receiving air from an inlet channel, holds the medicament in the fine powder form, and forms the starting section of an air passage; has a mean for generating turbulence when air flows through the airflow chamber such that, in use, the turbulent air flow causes vibration of a holder received by the airflow chamber to assist in releasing medicament contained within the holder; a positive pressure supplier switched on-off with a hand that connects to the airflow chamber to trigger and drive the powder flow through the air passage towards mouthpiece; a closure means or mechanism that closes the chamber to form an air passage: a sharp mean placed in the airflow chamber to pierce the holder of medicament to release it into the air passage; an outlet air channel that connects to the chamber to form a part of the air passage with or without one or more vents; a mouthpiece that is a part of the air passage through which the medicament can be delivered to a patient.

2. A hand-actuated positive pressure dry powder inhaler (PP-DPI) for administering an inhalable medicament, comprising: a chamber for receiving the wrapped or capsuled medicament in the fine powder form and for forming the starting section of an air passage; a closure means or mechanism for closing the chamber, the closure means or mechanism being moveable relative to the chamber; piercing means suitable for piercing the capsule; wherein movement of the closure means or mechanism relative to the chamber form a closing chamber; piercing means equipped in the chamber; an one-way air inlet and an air outlet defining an air passage therebetween, the air passage comprises one or more vents; and a holder for the capsule comprising: a chamber suitable for receiving the capsule; and means for generating turbulence in a fluid flow through the chamber such that, in use, the turbulent fluid flow causes vibration of a capsule received by the chamber so as to assist in releasing medicament contained within the capsule, wherein the means for generating turbulence extends substantially the entire length of the chamber and the inhalation passage; an air supplier with a switch by hand to provide positive pressure to the airflow chamber to trigger, drive and increase the turbulent flow to enhance the releasing medicament contained within the capsule to move easily towards the mouthpiece, which makes it easier and more efficient to, administer the medicament to a patient.

3. A hand-actuated positive pressure dry powder inhaler (PP-DPI) for administering an inhalable medicament, comprising: an airflow chamber and an air passage connected by an integrally mounded hinge configured to hinge the two parts of the inhaler from an open position to a closed position; the inhaler further comprising a dose to be inhaled which is sealed in the at least part of the airflow chamber by a frangible membrane and being arranged such that the frangible membrane is broken by an action of closing the inhaler prior to use and the dose is exposed to an entraining airflow in the circulating airflow chamber upon inhalation, and wherein the airflow chamber having air and entrained substance particles circulate further comprises an axis and being elongate along the axis, an one-way air inlet, a closed base and a vortex finder, the chamber being shaped wherein at least a part of the chamber decreases in cross-sectional area in a direction away from the one-way air inlet; wherein the one-way air inlet is configured to direct air to circulate around the periphery of the chamber, and the vortex, finder is configured such that air exits therethrough after a reversal of axial direction at the base of the chamber to set up a reverse flow cyclone in the chamber wherein air circulates in two generally concentric overlapping columns in opposite axial directions: an air supplier with a switch by hand to provide positive pressure to the airflow chamber to trigger and drive the medicament contained within the frangible membrane to flow easily towards the mouthpiece, which makes it easier and more efficient to administer the medicament to a patient.

4. A hand-actuated positive pressure dry powder inhaler (PP-DPI) for administering an inhalable medicament, comprising: an one-way air inlet channel through which air enters the inhaler; an airflow chamber that receives air from the inlet channel, the airflow chamber containing a single actuator to which a powdered medicament is adhered, wherein the airflow chamber has a longitudinal axis, and wherein the inlet channel is generally co-axial with the longitudinal axis; a retaining member disposed at an end of the chamber opposite the inlet channel, the retaining member having one or more openings sized to permit air and the powdered medicament to pass through the retaining member, and to prevent the actuator from passing through the retaining member; and an outlet channel through which air and the powdered medicament leave the inhaler through mouthpiece, wherein the outlet channel is generally co axial with the longitudinal axis of the airflow chamber; wherein the geometry of the inhaler is such that a flow profile is generated within the airflow chamber that causes the single actuator to repeatedly oscillate generally co-axially with the longitudinal axis, thus, by virtue of the repeated oscillations, detaching the, powdered medicament from the surface of the actuator to be entrained by the air and flow towards the mouthpiece; an air supplier with a switch by hand to provide positive pressure to the airflow chamber to trigger and drive medicament to flow towards the mouthpiece, which makes it easier and more efficient to administer the medicament to a patient.

5. A hand-actuated positive pressure dry powder inhaler (PP-DPI) for administering an inhalable medicament, comprising: an airflow chamber; a dose container disk rotatably secured within the airflow chamber, wherein the dose container disk comprises opposing upper and lower primary surfaces, a first row of circumferentially spaced apart through apertures associated with dose containers at a first radius and a second row of circumferentially spaced apart apertures associated with dose containers at a second radius; a first flexible sealant residing over the apertures in the upper surface, and a second flexible sealant residing over the apertures in the lower surface, and wherein the dose containers have dry powder therein; and a piercing mechanism configured to serially alternate between rows to pierce the scalants of a respective dose container in the first row, then pierce the sealants of a respective dose container in the second row, wherein the piercing mechanism comprises: a rotatable drum; and an elongate piercing member operably associated with the rotatable drum and capable of reciprocal movement between piercing and non-piercing positions; an air supplier with a hand switch to provide positive pressure to the airflow chamber to trigger and facilitate the dry powder flow easily towards the mouthpiece which makes it easier and more efficient to administer the medicament to a patient.

6. A hand-actuated positive pressure dry powder inhaler (PP-DPI) for administering an inhalable medicament, having (1) an air pump which is a hand-held squeezable bulb with an one-valve which permits the air flow into the airflow chamber only, and not suck the powder medicament out of the chamber; and/or the second one-way valve in the bulb which permits air only enter into the squeezable bulb to generate positive pressure repeatedly; or, (2) an air pump that is powered with a battery or plug-in electricity with an onioff switch; or (3) an air-pressure-filled container from which the positive pressure can be released upon turning on the switch of the container and the pressured air forcefully flows into the airflow chamber to facilitate the fine powder to flow through the air passage for easy inhalation by a patient.

7. A hand-actuated positive pressure dry powder inhaler (PP-DPI) for administering an inhalable medicament, having a positive pressure supplier that is a new addition to a negative pressure dry powder inhaler which either holds a single dose or multiple doses of the medicament, to change that negative pressure dry powder inhaler to become a positive pressure dry powder inhaler for consistently administering a dry powder medicament to a patient.

8. A hand-actuated positive pressure dry powder inhaler (PP-DPI) for administering an inhalable medicament, which is a stationary device at which a medical professional can help a patient to administer a therapeutic powder. or the PP-DPI is a portable device by which a patient can self-administer a therapeutic powder wherever and whenever it is needed.

9. A hand-actuated positive pressure dry powder inhaler (PP-DPI) for administering an inhalable medicament, which can supply the air to the DPI at the air flow rate ranging from about 1 to, about 120 liters per minute; preferably at 10 to 90 liters per minutes, and more preferably at 30 to 60 liters per minute.

10. An airflow adaptor of a hand-actuated dry powder inhaler which connects the air supplier under the positive pressure to a dry powder inhaler to change the negative pressure dry powder inhaler (NT-DPI) to a positive pressure dry powder inhaler (PP-DPI) comparing a conduit having an inlet end and an outlet end, wherein the inlet end allows air communication from an air supplier to the airflow chamber of a dry powder inhaler through the outlet end of the conduit when the switch of the air supplier is switched on to trigger and drive the powder to flow towards the mouthpiece cooperated by a patient to start inhalation.

11. A hand-actuated positive pressure dry powder inhaler (PP-DPI) for administering an inhalable medicament, having an airflow adaptor made in a one-size-fit-all design to secure any air supply under the positive pressure to any air passage of the dry powder inhaler without leakage. The airflow adaptor is made with elastic material or hard material or in any combination.

12. An air-supplier for supplying positive pressure to a hand-actuated positive pressure dry powder inhaler (PP-DPI) comprising: (1);a power supplier switched on/off with a hand, which is (a) an electronic power supplier; or (b) an elastic bulb onto which to apply pressing power by a human hand, or (c) an air-pressure-filled container to release positive pressure upon turning on by a hand; (2) a mechanical apparatus including an air pressure generator and an air passage; (3) a valve mean; (4) an airflow adaptor switched on/off by a hand to connect to the airflow chamber of the positive pressure dry powder inhaler; (5) a switch mean; (6) an optional meter or apparatus to control, measure and indicate air pressure for accurately supplying positive pressure to the airflow chamber of the positive pressure dry powder inhaler.

13. A hand-actuated positive pressure dry powder inhaler (PP-DPI) for administering an inhalable medicament, having a power supplier that supplies positive air pressure to the PP-DPI to have an air flow rate in the range of 5 to 105 liters/minute. Preferably, the power supplier supplies positive air pressure to the PP-DPI to have an air flow rate in the range of 15 to 90 liters/minute. More preferably, the power supplier supplies positive air pressure to the PP-DPI to have an air, flow rate in the range of 30 to 60 liters/minute.

14. A hand-actuated positive pressure dry powder inhaler (PP-DPI) for administering an inhalable medicament, having a power supplier that supplies positive air pressure to the PP-DPI to deliver constantly the positive air through the air inlet to the airflow chamber of the PP-DPI in the range of 100 to 4000 ml in one second. Preferably, the power supplier supplies positive air pressure to the PP-DPI to deliver constantly positive air via the air inlet to the PP-DPI airflow chamber in the range of 300 to 1000 ml in one second. More preferably, the power supplier supplies positive air pressure to the PP-DPI to deliver constantly positive air via the air inlet to the PP-DPI airflow chamber in the range of 300 to 600 nil in one second. More preferably, the power supplier supplies positive air pressure to the PP-DPI to constantly deliver positive air through the air inlet to the PP-DPI airflow chamber in the exact volume of 500 ml in one second.

15. A modern therapeutic kit comprising (1) a hand-actuated positive pressure dry powder inhaler (PP-DPI) for easy administration of the inhalable medicament packed in a single dose or in multiple doses, which comprises: an airflow chamber for receiving the wrapped or capsuled medicament in the fine powder form and for forming the starting section of an inhalation passage; a positive pressure supplier switched by a hand and connected to the airflow chamber which is also the starting section of the inhalation passage; a closure:means or mechanism for closing the airflow chamber; an inhalation passage comprises one or more vents; a group of the sharp means placed in the airflow chamber to pierce the wrap/capsule to release the medicament into the inhalation passage; a mouthpiece as a part of an, inhalation passage; and (2) an inhalable medicament in a dose container comprises a dry powder having a pharmaceutically active agent for treating a human disease/disorder, wherein the pharmaceutically active agent is in the fine powder form varying its particle size from 1 to 50 micron and is in a small amount (0.01 to 550 mg, preferably 0.10 to 350 mg) which, is accounted for 0.1% to 100% of the total formulation weight, and wherein the agent is a safe and effective medicine used to or to be used to treat a human disease, such as: abacavir for I-IV infection, acetaminophen and/or propoxyphene for arthritis; aclidinium, budesonide, formoterol, roflumilast, umeclidinium, vilanterol for COPD; adalimumab, leflunomide, tocilizumab for rheumatoid arthritis; albuterol, montelukast, methylprednisolone, mometasone/formoterol, or ipratropium for asthma; almotriptan, diclofenac, eletriptan for migraine alprazolam, amitriptyline, bupropion, fluoxetine, vilazodone for depression; alprostadil, arginine, avanafil, dextromethorphan, sildenafil, tadalafil, testosterone, or vardenafil, for erectile dysfunction; alteplase, aspirin, clopidogrel for stroke; amifostine, chlorambucil, cyclophosphamide, fluorouracil, or vincristine for cancer; amitriptyline for depression; amlodipine besylate for hypertension; amoxicillin, ceftriaxone, ciprofloxacin for urinary tract infection; amphetamine sulfate for attention deficit hyperactivity disorder; aripiprazole, lurasidone, risperidone, ziprasidone for schizophrenia; atenolol for angina;

atorvastatin calcium for osteoarthritis; atorvastatin, ezetimibe, pitavastatin, rosuvastatin, simvastatin for high cholesterol; azithromycin, levofloxacin, moxifloxacin for bronchitis; budesonide, sulfasalazine for inflammatory bowel disease; bupropion hydrochloride for depression; candesartan cilexetil for hypertension; carisoprodol for pain; ceftazidime for endocarditis; Celecoxib, Meloxicam, naproxen/esomeprazole for osteoarthritis; cetirizine, fluticasone furoate for hayfever; chlorpheniramine/acetaminophen, diphenhydramine got colds & Flu; clonazepam, lamotrigine, lurasidone for bipolar disorder; conjugated estrogens, zoledronic acid for osteoporosis; diethylpropion, lorcaserin, methamphetamine, phentermine for weight control; diphenhydramine for allergy; disopyramide, dofetilide for arrhythmia donepezil for Alzheimer's; duloxetine for depression; emtricitabine/rilpivirine/tenofovir for AIDS/HIV; enalapril, nebivolol, olmesartan, triamterene/hydrochlorothiazide for hypertension; epinephrine, epinephrine isomers, ephedrine for asthma and COPD; escitalopram for anxiety; esameprazole for GERD (Heartburn); estazolam, flurazepam. olanzapine for insomnia; esterified estrogens/methyltestosterone for menopause disorder; divalproex sodium ethosuximide, levetiracetam, primidone for seizures; ezetimibe for high cholesterol; fluoxetine for depressive disorders; gabapentin for nerve pain; guaifenesin for cough, heparin sodium for blood clots in the veins, arteries, or lungs; hydrochlorothiazide for edema; hydrocodone bitartrate for severe pain; hydromorphone, oxycodone, tapentadol for pain; hyoscyamine, mirabegron for urinary incontinence; ibuprofen for inflammation and pain; immune globulin for hepatitis A; insulin, insulin glargine, insulin lispro, pramlintide for diabetes (Type 1); insulin detemir, liraglutide, repaglinide, empagliflozin for diabetes (Type 2); isotretinoin for acne: levocetirizine for allergies; levofloxacin for bacterial infections; levothyroxine, liothyronine, thyroid for hypothyroidism; lisdexamfetamine for ADHD; loratadine or diphenhydramine for allergy; lorazepam for anxiety disorders; morphine, oxycodone for moderate to severe pain; naproxen for arthritis pain or inflammation; nifedipine for hypertrophic cardiomyopathy; oseltamivir, zanamivir for influenza; paroxetine hydrochloride for depression; pegloticase for gout; penicillin for bacterial infection; phentermine for obesity; pregabalin for fibromyalgia; pregabalin for seizures; quetiapine fumarate for schizophrenia, ranolazine for angina; risperidone for schizophrenia and symptoms of bipolar disorder; sertraline hydrochloride for depression; sildenafil citrate for pulmonary arterial hypertension; tratnadol hydrochloride for pain; valsartan for hypertension; vardenafil hydrochloride for impotence; venlafaxine hydrochloride for depression or zolpidem tartrate for sleep problems. The pharmaceutical active agent may be used in the form of salts, esters or solvates to thereby optimize the activity and/or stability of the medicament.

16. A modern therapeutic kit of claim 15 in which the dose of the pharmaceutically active agent administered with a hand-actuated PP-DPI is in a dose range of 0.01 mg to 350 mg to treat a disease or disorder as clinically justified or medically needed.

17. A therapeutic biologic kit comprising (1) a hand-actuated positive pressure dry powder inhaler (PP-DPI) for easy administration of the inhalable medicament packed in a single dose or in multiple doses, which comprises: an airflow chamber for receiving the wrapped or capsuled medicament in the fine powder form and for forming the starting section of an inhalation passage;

a positive pressure supplier switched onioff by hand connected to the airflow chamber, which is also the starting section of the inhalation passage; a closure means or mechanism for closing the airflow chamber; an inhalation passage comprises one or more vents; a group of the sharp means placed in the airflow chamber to pierce the wrap/capsule to release the medicament into the inhalation passage; a mouthpiece as a part of an inhalation air passage; and (2) an inhalable biologic agent in a dose container comprises a dry powder having a pharmaceutically active biologic agent for treating a human disease or disorder, wherein the pharmaceutically active biologic agent is in the fine powder form varying its particle size from 1 to 50 micron and is in a small amount (0.01 to 350 mg) which is accounted for 0.01% to 99.9% of the total formulation weight. Any biologics which can be delivered in the pulmonary route can be delivered by using a hand-actuated PP-DPI, such as 90Y-Ibritumomab tiuxetan, Abatacept. Abciximab, Adalimumab, ado-trastuzumab emtansine, Aflibercept, Agalsidase beta, Albiglutide, Aldesleukin, Alefacept, Alemtuzumab, Alemtuzumab, Algiucosidase alfa, Alteplase, Anakinra, asparaginase Erwinia chrysanthem, Basiliximab, Becapiermin, Belatacept, Belimumab, Bevacizumab, Bortezomib. Brentuximab vedotin, Canakinumab, Capromab, Pendetide, Certolizumab pegol, Cetuximab, Collagenase, Collagenase Clostridium Histolyticum, Daclizumab, Darbepoetin alfa, Dasatinib, Denileukin diflitox, Denosumab, Dornase alfa, Drotrecogin alfa, Dulaglutide, Ecallantide, Eculizumab, Efalizumab, Elosulfasc alfa, Epoetin alfa, Erlatinib, Etanercept, Everolimus, Fanolesomab, Filgrastim, Galsulfase, Gefitinib, Gemtuzumab, Glucarpidase, Golimumab, Hepatitis b vaccine; Ibritumomab tiuxetan, Idursulfase, Imatinib, Infliximab, Interferon alfa-2a, Interferon alfa-2b, Interferon alfa-2b, Interferon alfacon-1, Interferon alfa-n3, Interferon alpha, Interferon beta-1a, Interferon Beta-1b, Interferon gamma-1b, Interleukin-2, Ipilimumab, Lapatinib, Laronidase, Lenalidomide, Methoxypolyethylene glycol epoetin beta, Metreleptin, Muromonab-CD3, Natalizutnab, Nofetumomab, Obinutuzumab, Ocriplasmin, Ofatutnumab, Omalizumab, Oprelvekin, Palifermin, Palivizumab, Palivizumab, Panitumumab, Pegaspargase, Pegfilgrastim, Peginterferon alfa-2a, Peginterferon alfa-2b, Peginterferon beta-1a, Pegloticase, Pembrolizumab, Pertuzumab, Raniucirumab, Ranibizumab, Rasburicase, Raxibacumab, Reteplase, Rilonacept, Rituximab, Sargramostim, Siltuximab, Sorafenib, Sunitinib, tbo-filgrastim, Tenecteplase, Thalidomide, Tocilizumab, Tositumomab, Trastuzumab, Ustekinumab, Vedolizumab, Vermurafenib, or ziv-aflibercept.

18. A therapeutic biologic kit in which the dose of the pharmaceutically active biological agent administered with a hand-actuated PP-DPI is in a dose range of 0.001 mg to 350 mg to treat a human disease or disorder as clinically justified or medically needed.

19. A natural therapeutic kit to treat a human disease or disorder in the fine powder form for being administered with a hand-actuated positive pressure dry powder inhaler (PP-DPI) or a negative pressure dry powder inhaler (NP-DPI) varying its particle size from 1 to 50 micron and in a small amount (0.1 to 350 mg) which is accounted for 0.01% to 99.9% of the total formulation weight, comprising one or more extracts from (1) a commonly used herb in the Traditional Chinese Medicine, such as: Aloe vera (Lu Hui), Astragalus Root (Huang Qi), Codonopsis Mang Shen), Belladonnia alkaloids (Dian Qie Jian), Cordyceps militaris (Dong Chong Xia Cao), Cortex Mori Albae Radicis (Sang Bai Pi), egg shell, Flos Tussilaginis Farfarae (Kean-Dong-Hua), Fructus Corni Officinalis (Shan Zhu Yu), Fructus Perilla Frutescens (Su Zi), Ginseng (Ren Shen), Glycyrrhizae (Gan Cao), Herba Ephedrae (Ma Huang), Jujube (Da Zao), Nonglutinous Rice (Geng Mi), Ophiopogonis (Mai Men Dong), Peppermint (Bo He), Pinelliae Preparatum (Zhi Ban Xia), Radix Dioscoreae Oppositae (Shan Yao), Radix Rehmanniae Preparata (Shu Di Huang), Radix Scutellariae (Huang Qin), Rhizoma Alismatis Orientalis (Ze Xie), Sclerotium Poriae Cocos (Fu Ling), Semen Pruni Armeniacae (Xing-Ren), Sophora flavescens ait (Ku Shen), Tripterygium wilfordii (Lei Gong Teng), or Tuber Pineilia (Ban Xia), or (2) a formula used in the Traditional Chinese Medicine, such as: An Zhong San (Calm the Middle Powder), Ba Wei Di Huang Wan (Eight-Ingredient Pill with Rehmannia), Ba Dien Tang (Eight-Treasure Decoction), Bai He Gu Jin Wan (Lilium Teapills), Ban Xia Bai Zhu Tian Ma Tang (Pinellia, Atractylodes Macrocephala, and Gastrodia Decoction), Ban Xia Xie Xin Tang (Pineilia Decoction to Drain the Epigastrium), Bao Chan Wu You Fang (Preserve Pregnancy and Care-Free Decoction), Cang Er Zi (Upper Chamber Teapills), Chai Ge Jie Ji Tang (Bupleurum and Kudzu Decoction to Release the Muscle Layer), Chai Hu Gui Zhi Tang (Bupleurum and Cinnamon Twig Decoction), Chai Hu Qing Gan Tang (Bupleurum Decoction to Clear the Liver), Dang Gui Bu Xue Tang (Tangkuei Decoction to Tonify the Blood), Dang Gui Jing (Tang Kwei Gin), Dang Gui Liu Huang Tang (Tangkuei and Six-Yellow Decoction), Dang Gui Nian Tong Tang (Tangkuei Decoction to Lift the Pain), Dang Gui Shao Yao San (Tangkuei and Peony Powder), Dun Sou San (Long-Bout Cough Powder), Er. Zhu Tang (Two-Atractylodes Decoction), Gan Lu Yin (Sweet Dew Decoction), Ge Gen Huang Qin Huang Lian Tang (Kudzu, Coptis, and Scutellaria Decoction), Gua Lou Zhi Shi Tang (Trichosanthes Fruit and Immature Bitter Orange Decoction), Gui Qi Jian Zhong Tang (Tangkuei and Astragalus Decoction to Construct the Middle), Gui Zhi Tang (Cinnamon Twig Teapills), Huai Hua San (Sophora Japonica Flower Powder), Huang Qi Jian Zhong Tang (Astragalus Decoction to Construct the Middle), Ling Gui Zhu Gan Tang (Poria, Cinnamon Twig, Atractylodes Macrocephala, and Licorice Decoction), Liu Wei Di Huang Wan (Six Flavor Teapills), Ma Huang Xing Ren Gan Cao Shi Gao Tang (Ephedra, Apricot Kernel, Licorice, and Gypsum Decoction), Mai Men Dong Tang (Ophiopogonis Decoction), Qiang Huo Sheng Shi Tang (Notopterygium Decoction to Overcome Dampness), Qing Bi Tang (Clear the Nose Decoction), Qing Fei Tang (Clear the Lung Decoction), Qing FIao Bie Jia Tang (Artemisia Annua and Soft-Shelled Turtle Shell Decoction), Qing Shang Fang Feng Tang (Clear the Upper and Guard the Wind Decoction), Qing Shu Yi Qi Tang (Clear Summer-Heat and Augment the Qi Decoction), Qing Xin Li Ge Tang (Clear the Epigastrium and Benefit the Diaphragm Decoction), Qing Xin Lian Zi Yin (Lotus Seed Decoction to Clear the Heart), Qing Zao Jiu Fei Tang (Eliminate Dryness and Rescue the Lung Decoction), Ren Shen Yang Ying Tang (Ginseng Decoction to Nourish the Nutritive Qi), San Huang Xie Xin Tang (Three-Yellow Decoction to Sedate the Epigastrium), San Zhong Kui Jian Tang (Disperse the Swelling and Break the Hardness Decoction), Shao Yao Tang (Peony Decoction), Shi Liu Wei Liu Qi Yin (Sixteen-Ingredient Decoction to Flow Qi), Shou Wu Zhi (Shou Wu Essence), Shu Jing Huo Xue Tang (Relax the Channels and Invigorate the Blood. Decoction), Si Jun Zi Tang (Four Gentlemen Teapills), Si Ni San (Four Pillars Teapills), Si Wu Tang (Four Substances For Women), Tao He Cheng Qi Tang (Peach Pit Decoction to Order the Qi), Wei Ling Tang (Calm the Stomach and Poria Decoction), Wen Qing Yin (Warming and Clearing Decoction), Wu Zhu Yu Tang (Evodia Decoction), Xiang Sha Ping Wei San (Cyperus and Amomum Powder to Calm the Stomach), Xiao Chai Hu Tang (Minor Bupleurum Decoction), Xiao Cheng Qi Tang (Minor Order the Qi Decoction), Xie Huang San (Drain the Yellow Powder). Xin Yi Qing Fei Yin (Magnolia Decoction to Clear the Lung), Xue Fu Zhu. Yu Tang (Stasis In The Mansion Of Blood Teapills), Yang Xin Tang (Nourish the Heart Decoction), Yi Gan San (Restrain the Liver Powder), Yin Chen Hao Tang (Artemisia Scoparia Decoction), Yin Qiao San (Honeysuckle and Forsythia Powder), Yu Ping Feng San (Jade Screen Teapills), Yu Nu Jian (Jade Woman Decoction), Zhe Chong Yin (Break the Conflict Decoction), Zhi Gan Cao (Honey-fried Liquorice Root), Zhi Sou San (Stop Coughing Powder), Zhu Ling Tang (Polyporus Decoction), Zhu Ye Shi Gao Tang (Bamboo Leaves and Gypsum Decoction), or Zi Yin Jiang Huo Tang (Nourish Yin and Descend the Fire Decoction).

20. A natural therapeutic kit to treat a human disease or disorder in the fine powder form in which a human disease or disorder is any disease or disorder as clinically justified and medically needed, such as: an endocrine disease, a gastrointestinal disease, a genetic disorder, a neurological disorder, voice disorder, an adrenal disorder, an allergic disorder, an anxiety disorder, an articulation disorder, an autonomic nerve disorder, an acute stress disorder, a balance disorder, a behavioral disorder, a bleeding disorder, a bipolar disorder, a cartilage disorder, a clotting disorder, a connective tissue disorder, a depressive disorder, a disc disorder, a digestive disorder, an eating disorder, a female genital disorder, a hearing disorder, an immune disorder, a metabolic disorder, a mood disorder, a nervous system disorder, a neuronal migration disorder, a neurological disorder, an orthopedic disorder, a personality disorder, a psychiatric disorder, a psychoactive substance abuse disorder, a psychological disorder, a sexual dysfunction, a sleep disorder, a social anxiety disorder, a soft tissue disorder, a spinal cord disorder, a testicle disorder, a thymus disorder, a thyroid disorder, or a vein disorder.

21. A combinational therapeutic kit including a hand-actuated positive pressure dry powder inhaler (PP-DPI) for easy administration of the inhalable medicament packed in a single dose or in multiple doses for treating a human disease or disorder comprises an airflow chamber for receiving the wrapped or capsuled medicament in the fine powder form and for forming the starting section of an inhalation passage; a positive pressure supplier with a hand-switch connected to the airflow chamber which is also the starting section of the inhalation passage; a closure means or mechanism for closing the airflow chamber; an inhalation passage comprises one or more vents; a group of the sharp means placed in the housing/chamber to pierce the wrap/capsule to release the medicament into the inhalation passage; a mouthpiece as a part of an inhalation passage; and (2) an inhalable medicament in a dose holder which comprises a dry powder having a pharmaceutically active agent, such as albuterol, and wherein the agent can be used in the form of salts, esters or solvates to thereby optimize the activity and/or stability of the medicament; and/or (3) an inhalable medicament in a dose holder comprises a dry powder having a pharmaceutically active biological agent in a dose range of 0.01 to 350 mg, such as Alemtuzumab; and/or (4) one or more of the natural extracts in the fine powder form in a small amount (0.1 to 350 mg) from a herb or a mix (formula), such as Astragalus Root (Huang Qi), or Zi Yin Jiang Huo Tang (Nourish Yin and Descend the Fire Decoction), (5) with or without any excipient.

22. A combinational therapeutic kit having an inhalable medicament in the fine powder form, and at last two active ingredients which are with the particle sizes from 1 to 100 micron, and is in a small amount (0.001 mg to 350 mg); and wherein the inhalable medicament is homogenized with or without an excipient to have an acceptable homogeneity defined as the variation of medicament is within the range of +/−20% from the target mean.

23. A combinational therapeutic kit in which any human disease or disorder can be treated by using the combinational therapeutic kit, such as acute bronchitis, acute respiratory distress syndrome (ARDS), asthma, acute or chronic bronchiolitis, bronchopulmonary dysplasia, chronic bronchitis, COPD, cystic fibrosis, emphysema, hantavirus pulmonary syndrome, hypersensitivity pneumonitis, influenza, lung cancer, pneumonia, primary pulmonary hypertension, pulmonary arterial hypertension, pulmonary fibrosis, pulmonary vascular disease, respiratory syncytial virus infection, severe acute respiratory syndrome, sleep apnea, tuberculosis, common cold, sinusitis, allergic rhinitis, stridor, tonsillitis, epiglottitis, whooping cough (Pertussis), croup, cancer, heart disease, hypertension, diabetes, obesity, a mental disorder (depression), a skeletomuscular disorder (back pain), arthritis, hyper or, hypothyroid disorder, sleep disorder, infection caused by a microorganism, immunity disorder (allergy), or any other disease or disorder as clinically justified or medically needed.

24. A method of using a natural therapeutic kit containing a hand-actuated positive pressure dry powder inhaler (PP-DPI) and an inhalable therapeutic natural powder, or using a modern therapeutic kit containing a hand-actuated PP DPI and an inhalable pharmaceutically active ingredient, or using a therapeutic biologic kit containing a hand-actuated PP-DPI and an inhalable pharmaceutically active biologic powder, or using a combinational therapeutic kit containing a hand-actuated PP-DPI and an inhalable powder of the combinational mix, comprising: (1) load or make sure the inhalable medicament is loaded in the airflow chamber of the hand-actuated PP-DPI: (2) pierce the wrap or capsule containing the medicament; (3) place the mouthpiece inside of the mouth after a deep exhalation; (4) use hand to turn on the supplier of the positive pressure to the airflow chamber to trigger the release of the medicament; (5) start a gentle inhalation at the same time of turning on the positive pressure supplier; and (6) rinse mouth and clean mouthpiece for'next use.

25. A therapeutic kit for relieving symptoms of a respiratory disease comprising: (1) an airflow chamber that receives air from the inlet, channel, holds the capsule containing medicament in the fine powder form, and forms the starting section of an air passage; has a mean for generating turbulence when air flows through the airflow chamber such that, in use, the turbulent air flow causes vibration of a capsule received by the airflow chamber to assist in releasing medicament contained within the capsule; (2) closure means that closes, the airflow chamber to form an, air passage: (3) a pair of sharp mean placed in the airflow chamber to pierce the holder_{<}f medicament to release it into the air passage; (4) an outlet air channel that connects to the chamber to form a part of the air passage with or without one or tnore vents; (5) a mouthpiece that is a part of the air passage through which the medicament can be delivered to a patient; and (6) a pack of capsules containing the medicament in relieving symptoms of a respiratory disease.

26. A therapeutic kit in which the medicament is a bronchodilator such as epinephrine, an anti-inflammation agent such as corticosteroid, an antihistamine agent, such as loratadine or diphenhydramine, an anti-viral agent, such as abacavir, a mucous thinner, such as guaifenesin, or any combination of at least two of these functional agents, with or without a natural therapeutic agent, such as licorice

It is to be understood that numerous workable combinations of features and elements of the PP-DPTs, treatment mixes and therapeutic kits can be implemented according to various embodiments of the invention disclosed herein. Practical implementations of the invention are numerous and not limited to the detailed embodiments and descriptions provided here, which are for the purpose of clarity and to enable the making of numerous new therapeutic kits and other products by practitioners skilled in the art.

As will be understood by one skilled in the art, for any and all purposes, such as in terms of providing a written description, all ranges disclosed in this disclosure also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed in this disclosure can be readily broken down into a lower third, middle third and upper third, etc. As will also be understood by one skilled in the art all language such as “up to,” “at least” and the like include the number recited and refer to ranges which can be subsequently broken down into subranges as discussed above. Finally, as will be understood by one skilled in the art, a range includes each individual member.

From the foregoing, it will be appreciated that various embodiments of the present disclosure have been described for purposes of illustration, and that various modifications may be made without departing from the scope and spirit of the present disclosure. Accordingly, the various embodiments disclosed are not intended to be limiting, with the true scope and spirit being indicated by the following claims.

1. A positive pressure dry powder inhaler for administering an inhalable medicament, comprising:
an airflow chamber (8) for receiving air from an inlet channel and holding the medicament in the fine powder form, the airflow chamber (8) forming a starting section of an air passage (4), the airflow chamber (8) including a turbulence generating mechanism that generates turbulent air flow through the airflow chamber such that during use of the hand-actuated positive pressure dry powder inhaler, the turbulent air flow causes vibration of a holder (31) received by the airflow chamber (8) to assist in releasing the medicament contained within the holder;
a positive pressure supplier (2), configured to switch on-off, connected to the airflow chamber for triggering and driving the medicament through the air passage towards a mouthpiece (6);
a closure means (3.1, 3.2) that closes the airflow chamber (8) to form an air passage;
a piercing element (1) coupled to the airflow chamber (8) to pierce the holder of medicament and release the medicament into the air passage;
an outlet air channel that connects to the airflow chambe (8) to form a part of the air passage with or without one or more vents; and
wherein the mouthpiece (6) is formed with the air passage to deliver the medicament.

2. The positive pressure dry powder inhaler of claim 1, wherein the positive pressure supplier is an air pump comprising a squeezable bulb having a one-way valve, the one-way valve permitting air flow only into the airflow chamber without withdrawing the medicament out of the chamber.

3. The positive pressure dry powder inhaler of claim 1, further comprising a second one-way valve which permits air to only enter into the squeezable bulb to generate positive pressure

4. The positive pressure dry powder inhaler of claim 1, wherein the positive pressure supplier comprises an electrically air pump.

5. The positive pressure dry powder inhaler of claim 1, wherein the positive pressure supplier comprises an air-pressure-filled container from which the positive pressure can be released upon activation of a switch.

6. The hand-actuated positive pressure dry powder inhaler of claim 1, wherein the inhaler is configured to supply air at an air flow rate ranging from about 1 liters per minute to about 120 liters per minute.

7. The hand-actuated positive pressure dry powder inhaler of claim 1, wherein the inhaler is configured to supply air at an air flow rate ranging from about 10 liters per minute to about 90 liters per minute.

8. The hand-actuated positive pressure dry powder inhaler of claim 1, wherein the inhaler is configured to supply air at an air flow rate ranging from about 30 to 60 liters per minute.

9. The hand-actuated positive pressure dry powder inhaler of claim 1, further comprising an airflow adaptor configured to connect the air supplier under the positive pressure to a dry powder inhaler and change the negative pressure dry powder inhaler (NP-DPI) to a positive pressure dry powder inhaler (PP-DPI) by providing a conduit having an inlet end and an outlet end, wherein the inlet end allows air communication from an air supplier to the airflow chamber of a dry powder inhaler through the outlet end of the conduit when the switch of the air supplier is switched on to trigger and drive the dry powder to flow towards the mouthpiece.

10. The hand-actuated positive pressure dry powder inhaler of claim 1, wherein said inhalable medicament is a bronchodilator, such as short-acting β2-agonists (salbutamol, ephedrine or epinephrine); or long-acting β2-agonists (salmeterol xinafoate, formoterol); or anticholinergics (tiotropium, ipratropium bromide), or any combination of these agents for treating asthma and COPD.

11. The hand-actuated positive pressure dry powder inhaler of claim 1, wherein said inhalable medicament is insulin glargine, or insulin lispro. or pramlintide; or insulin detemir, or liraglutide, or repaglinide, or empagliflozin, for treating diabetes mellitus.

12. The hand-actuated positive pressure dry powder inhaler of claim 1, wherein said inhalable medicament is arginine, avanafil, dextromethorphan, sildenafil, tadalafil, testosterone, or vardenafil, an extract from Epimedium brevicornu Maxim, Herba cynomorii, Cistanche deserticola Ma, or Wedelia chinensis, alone, or the mix of any of these agents, with or without a carrier, such as inhalable lactose, for treating erectile dysfunction.

13. The hand-actuated positive pressure dry powder inhaler of claim 1, wherein said inhalable medicament is chemical, such as ibuprofen; or a biological agent, such as Adalimum.ab, or an extract from an herb, such as Codonopsis, used alone, or used in the mix of any of these agents, with or without a carrier, such, as inhalable lactose, for treating a human disease. such as arthritis.

14. The hand-actuated positive pressure dry powder inhaler of claim 1, wherein said inhalable medicament comprises a dry powder having a pharmaceutically activity for treatment of a human disease, and the fine powder comprises particles that vary in particle size with the particle size ranging from about 1 micron to about 50 microns, in a range of 0.001 to 300 mg which accounts for 0.1% to 100% of the total formulation weight.
